(19) United States
(12) Patent Application Publication
(10) Pub. No.: US 2026/0364203 A1
(43) Pub. Date: Nov. 19, 2026

(54) AAV-CFTR VECTORS AND METHODS OF USING SAME

(71) Applicant: Children's Hospital Medical Center, Cincinnati, OH (US)

(72) Inventors: Yutaka Maeda, Cincinnati, OH (US); John Brewington, Independence, KY (US); Alicia Ostmann, Cincinnati, OH (US)

(21) Appl. No.: 19/487,086

(22) PCT Filed: Jun. 12, 2024
(86) PCT No.: PCT/US2024/033546

§ 371 Date: Nov. 24, 2025

Related U.S. Application Data
(60) Provisional application No. 63/472,410, filed on Jun. 12, 2023.
(60) Provisional application No. 63/535,873, filed on Aug. 31, 2023.

(51) Int. Cl. A61K 48/00; A61K 38/17; A61P 11/00

(52) U.S. Cl. CPC A61K 48/005; A61K 38/1709; A61K 48/0033; A61P 11/00

(57) ABSTRACT

Disclosed are paired nucleic acid construct systems for the delivery of a human cystic fibrosis transmembrane conductance regulator (CFTR) protein to an individual in need thereof. The two-part system comprises a first construct comprising a first portion of a human CFTR gene fused to Rhodothermus marinus intein-N cDNA (CFTR-N-inteinN) and a second construct comprising a second portion of a human CFTR gene fused to Rhodothermus marinus intein-C cDNA (inteinC-CFTR-C). Further disclosed are methods of treating an individual having cystic fibrosis (CF) comprising administration of the paired nucleic acid constructs.

### CROSS-REFERENCE TO RELATED APPLICATIONS

This application claims priority to and benefit of U.S. Provisional Application Ser. No. 63/472,410, filed Jun. 12, 2023, and U.S. Provisional Application Ser. No. 63/535,873 filed Aug. 31, 2023, the contents of which are incorporated in their entirety for all purposes.

### STATEMENT REGARDING FEDERALLY-SPONSORED RESEARCH

This invention was made with government support under K08HL144825, R01CA240317, and U01HL134745 awarded by the National Institutes of Health. The government has certain rights in the invention.

### REFERENCE TO SEQUENCE LISTING

A Sequence Listing submitted as an XML text file via EFS-WEB is hereby incorporated by reference. The name of the XML file for the Sequence Listing is 2022-1201c_SequenceList.xml, the date of the creation of the XML file is Jun. 11, 2024, and the size of the XML file is 43,051 bytes.

### BACKGROUND

Cystic Fibrosis (CF) is a lethal genetic disease resulting in multiorgan dysfunction that is caused by loss of function for the anion channel CFTR. CFTR is a large gene, 1480 amino acids in length, a defect in which is the cause of cystic fibrosis (CF). Although the advent of highly effective CFTR modulator therapy (HEMT, e.g., elexacaftor/tezacaftor/ivacaftor) has significantly improved the care of many individuals with CF, about 10% of patients with CF carry CFTR mutations which are ineligible for these drugs. This includes those with class 1 mutations that produce no functional CFTR protein (e.g., G542X, R553X, W1282X). Gene therapy to replace defective CFTR is a promising approach for this cohort, as such therapy will restore CFTR function regardless of CFTR genotype. Such gene therapy however, has been pursued since the 1990s with limited success. Initial studies utilizing adenovirus were halted due to toxicity. Recently, adeno-associated virus (AAV, also known as parvovirus) has been clinically proven as a safe means to deliver therapeutic genes to diseased organs. (Wang D, Tai P W L, Gao G. Adeno-associated virus vector as a platform for gene therapy delivery. Nat Rev Drug Discov. 2019; 18(5):358-378. doi:10.1038/s41573-019-0012-9, Wang J H, Gessler D J, Zhan W, Gallagher T L, Gao G. Adeno-associated virus as a delivery vector for gene therapy of human diseases. Signal Transduct Target Ther. 2024; 9(1):78. Published 2024 Apr. 3. doi:10.1038/s41392-024-01780-w, Li C, Samulski R J. Engineering adeno-associated virus vectors for gene therapy. Nat Rev Genet. 2020; 21(4):255-272. doi:10.1038/s41576-019-0205-4.) While delivery of an intact CFTR gene using AAV vector technology would be advantageous in treating CF, the large size of the gene makes it difficult to package for delivery in vivo. That is, the limit of AAV vectors cargo capacity prevents development of AAV-based therapies that require delivery of genes with a coding sequence (CDS) larger than 4 kb; AAV can only carry a gene of about 4,000 basepairs or less. As the CFTR gene is 4,440 base pairs, this exceed the AAV size limitation. In an attempt to overcome this size limitation, portions of the CFTR gene (e.g., the N-terminus or part of the R domain) have previously been deleted, allowing the shortened CFTR gene to be packaged into AAV2 or AAV5. Although these studies reported that in vivo CFTR function was retained despite the deletions, clinical trials were not successful. Additionally, attempts have been made to deliver split CFTR mRNA using AAV2, AAV5, or AAV6.2, enabling spliceosome-mediated RNA trans-splicing and restoration of CFTR function, but were not validated in vivo.

Previous studies of AAV-based gene therapy in CF have been unsuccessful or incomplete for several reasons. An early phase clinical trial using AAV to deliver partial R domain-deleted CFTR demonstrated highly efficient delivery to lung tissues, however, this truncated CFTR protein did not restore CFTR function in patients.¹² Although this partial R domain-deleted CFTR was functional in human CF airway epithelial cells in vitro⁹ and in a CF pig model in vivo,¹⁰ there is a possibility that the CFTR R domain plays a critical functional role in human CF airway epithelial cells in vivo. In an attempt to deliver intact CFTR without such a deletion, Zhu et al. used intein technology to create split CFTRs fused to inteins from Synechocystis sp, which were successfully recombined and became more functional than split CFTRs without inteins in Baby Hamster Kidney fibroblast (BHK) cells.¹⁹ However, it is believed that these constructs have not been packaged into actual AAV or tested in CF patient cells to determine whether they can indeed rescue CFTR function.

Thus, there is a need for constructs and vectors that can be used for effective gene therapy for delivery of CFTR to treat CF.

### BRIEF SUMMARY

Disclosed are paired nucleic acid construct systems for the delivery of a cystic fibrosis transmembrane conductance regulator (CFTR) protein to an individual in need thereof. The two-part system may comprise a first construct comprising a first portion of a CFTR gene fused to intein-N cDNA (CFTR-N-inteinN) and a second construct comprising a second portion of a CFTR gene fused to intein-C cDNA (inteinC-CFTR-C). Further disclosed are methods of treating an individual having cystic fibrosis (CF) comprising administration of the paired nucleic acid construct systems.

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 depicts a schematic of intein technology to deliver CFTR to CF airway using AAV.

FIG. 2A depicts a schematic for the construct for an AAV6 carrying split CFTRs fused with inteins. This vector (N+C; Dual AAV6) restores the physiological functions of CFTR in F508del homozygous primary human bronchial epithelial (F508del HBE) cells. FIG. 2B: shown is immunofluorescence images indicating that Flag-tagged N-terminus portion of CFTR attached to intein N (intN) is co-expressed with HA-tagged C-terminus portion of CFTR attached to intein C (intC) in 293T cells. FIG. 2C: shown are western blots indicating split CFTRs and recombined CFTR are detected by Flag or HA antibody. N-terminus portion is detected by Flag or CFTR antibody, and C-terminus portion is detected by HA antibody in 293T human embryonic kidney cells 3 days after infection with AAV6 carrying CFTR-N-intN and intC-CFTR-C. FIG. 2D: CFTR activity was assessed in Ussing chambers. F508del primary HBE cells grown at air-liquid interface were mounted in Ussing chambers and short-circuit current was measured under voltage clamp conditions.

FIGS. 3A-3C demonstrate that AAV6.2FF (a variant of AAV6 serotype) carrying split CFTRs (N+C; dAAV) restores the physiological function of CFTR in primary nasal epithelial cells from people with CF (PwCF) who are insensitive to CFTR modulators. FIG. 3A: Nasal epithelial cells from PwCF who are insensitive to CFTR modulators were expanded in submerged culture in vitro, infected with AAV6.2FF carrying split CFTRs, and subsequently transferred to air-liquid interface culture. FIG. 3B and FIG. 3C: CFTR-dependent short-circuit current in nasal cells homozygous for c850dup (3B) or 1525-1G>A (C) CFTR, which are insensitive to Elexacaftor/Tezacaftor/Ivacaftor. Cells treated with dual AAVs demonstrated significant improvements in CFTR-specific current (cAMP+VX-770) compared to untreated cells.

FIG. 4 depicts in vitro analysis using human CF primary airway cells. In vitro analysis can be conducted 1) using nasal or bronchial airway cells isolated from CF patients, including patients who do not respond to CF modulators (e.g., elexacaftor/tezacaftor/ivacaftor). 2) isolated cells can be cultured in monolayer at air-liquid interface (ALI). 3) cultured differentiated nasal epithelial cells can be infected with AAV carrying split CFTRs. 4) CFTR function can be assessed in AAV infected cells (1) using Ussing Chambers (B). Mucocilliary clearance in the infected cells can be assessed (C&D). Immunofluorescence staining can be conducted to assess which cell types express recombined split CFTRs delivered by AAV (A&E). Swelling test, indicating CFTR function can also be conducted using spheroid culture for additional cells (F).

FIGS. 5A-5D depict in vivo mouse analysis that can be conducted using a CF-relevant model (ENaCs) mice that produce abundant mucus in the airways. FIG. 5A: shown is a construct that was used to create ENaCs transgenic mice. Rat CCSP promoter is active in club/secretory airway cells in mice. Mucus stained with Alcian blue was detected in trachea of the mouse. Shown image was obtained from a sacrificed ENaCs transgenic mouse three days after birth. Trachea region of the mouse was harvested and fixed by 4% paraformaldehyde. Fixed tissue was embedded in paraffin. Paraffin section was used for Alcian blue staining. FIG. 5B: ENaCP transgenic mice whose airway filled with mucus can be intratracheally infected with AAV carrying split CFTRs (a dual AAV). Months (up to a year) after infection mouse lungs can be harvested for assessing the expression of recombined CFTR by immunohistochemistry and western blotting. FIG. 5C: GFP expression in airways transduced intratracheally by AAV6.2 (a variant of AAV6 serotype) was higher than that by AAV6 in ENaCs transgenic mice. Black dots indicate GFP staining. Two months old ENaCp transgenic mice were intratracheally infected with AAV6 or AAV6.2 carrying GFP driven by CMV. Two weeks after infection, mice were sacrificed, and lungs were harvested and fixed by 4% paraformaldehyde. Fixed tissue was embedded in paraffin. Paraffin section was used for immunohistochemistry using 3,3′-diaminobenzidine (DAB) staining with GFP antibody (Cell Signaling Technology, cat #2956) FIG. 5D: GFP protein expression from left lungs of the indicated mice with or without transduction of AAV6.2 carrying GFP was assessed by western blotting. AAV6.2 carrying GFP was intratracheally injected into the indicated mice. One week after injection, left lungs were harvested, and protein extracted. Western blotting (IB) was performed using extracts with GFP (Cell Signaling Technology, cat #2956) and Actin (MilliporeSigma, cat #A2066) antibodies.

FIG. 6 depicts a cellular response in human nasal epithelial (HNE) cultures from an individual with cystic fibrosis (CF) without CFTR modulator access. The left panel shows representative short circuit current tracings, with vehicle-treated cells in solid lines and dual AAV (dAAV)-treated cells in dashed lines. The right panel indicates the patient's CFTR genotype and shows aggregate tracing data for CFTR function under control, modulator-treated, and dAAV-treated conditions. CFTR function >10% of wild-type is easily achieved. For all aggregate data, n=4 inserts. **p<0.01; ***p<0.001; ****p<0.0001 by one-way ANOVA with Dunnet's multiple comparisons test against control samples.

FIG. 7 depicts a cellular response in human nasal epithelial (HNE) cultures from an individual with cystic fibrosis (CF) without CFTR modulator access. The left panel shows representative short circuit current tracings, with vehicle-treated cells in solid lines and dual AAV (dAAV)-treated cells in dashed lines. The right panel indicates the patient's CFTR genotype and shows aggregate tracing data for CFTR function under control, modulator-treated, and dAAV-treated conditions. CFTR function >10% of wild-type is easily achieved. For all aggregate data, n=4 inserts. **p<0.01; ***p<0.001; ****p<0.0001 by one-way ANOVA with Dunnet's multiple comparisons test against control samples.

FIG. 8 depicts a cellular response in human nasal epithelial (HNE) cultures from an individual with cystic fibrosis (CF) without CFTR modulator access. The left panel shows representative short circuit current tracings, with vehicle-treated cells in solid lines and dual AAV (dAAV)-treated cells in dashed lines. The right panel indicates the patient's CFTR genotype and shows aggregate tracing data for CFTR function under control, modulator-treated, and dAAV-treated conditions. CFTR function >10% of wild-type is easily achieved. For all aggregate data, n=4 inserts. **p<0.01; ***p<0.001; ****p<0.0001 by one-way ANOVA with Dunnet's multiple comparisons test against control samples.

FIG. 9 depicts a cellular response in human nasal epithelial (HNE) cultures from an individual with cystic fibrosis (CF) without CFTR modulator access. The left panel shows representative short circuit current tracings, with vehicle-treated cells in solid lines and dual AAV (dAAV)-treated cells in dashed lines. The right panel indicates the patient's CFTR genotype and shows aggregate tracing data for CFTR function under control, modulator-treated, and dAAV-treated conditions. CFTR function >10% of wild-type is easily achieved. For all aggregate data, n=4 inserts. **p<0.01; ***p<0.001; ****p<0.0001 by one-way ANOVA with Dunnet's multiple comparisons test against control samples.

FIG. 10 depicts a cellular response in human nasal epithelial (HNE) cultures from an individual with cystic fibrosis (CF) without CFTR modulator access. The left panel shows representative short circuit current tracings, with vehicle-treated cells in solid lines and dual AAV (dAAV)-treated cells in dashed lines. The right panel indicates the patient's CFTR genotype and shows aggregate tracing data for CFTR function under control, modulator-treated, and dAAV-treated conditions. CFTR function >10% of wild-type is easily achieved. For all aggregate data, n=4 inserts. **p<0.01; ***p<0.001; ****p<0.0001 by one-way ANOVA with Dunnet's multiple comparisons test against control samples.

FIG. 11 depicts a cellular response in human nasal epithelial (HNE) cultures from an individual with cystic fibrosis (CF) without CFTR modulator access. The left panel shows representative short circuit current tracings, with vehicle-treated cells in solid lines and dual AAV (dAAV)-treated cells in dashed lines. The right panel indicates the patient's CFTR genotype and shows aggregate tracing data for CFTR function under control, modulator-treated, and dAAV-treated conditions. A statistically significant response to dAAV is observed. For all aggregate data, n=4 inserts. **p<0.01; ***p<0.001; ****p<0.0001 by one-way ANOVA with Dunnet's multiple comparisons test against control samples.

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting. The methods may comprise, consist of, or consist essentially of the elements of the compositions and/or methods as described herein, as well as any additional or optional element described herein or otherwise useful in the manufacture or use of human CFTR-Rhodothermus marinus intein constructs.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

While effective gene therapy techniques for delivery of an intact CFTR is highly desirable, such efforts, prior to Applicant's invention, have not been successful, such that a need for effective therapies remains in the art. One approach for delivery of CFTR into cells using AAV is “intein” technology, which auto-splices proteins together. This approach has been utilized in the CRISPR/Cas9 field to deliver two halves of the Cas9 gene (4,158 bp) attached to complementary inteins, reducing the genetic construct size to fit into two separate AAVs. Once in the cells, AAV-delivered Cas9 half-genes are transcribed, translated, and the resulting proteins recombined by intein-mediated protein ligation to form a complete Cas9 protein.^{15,16} This strategy circumvents the size limitation of AAV, and so can be adapted to deliver CFTR (4,440 bp). In this approach, the N-terminal portion of CFTR cDNA is fused to intein-N cDNA, and the C-terminal portion is fused to intein-C cDNA, and each are packaged into separate AAVs. After AAV infection, each gene produces the coded half-protein, and the two portions of CFTR protein are recombined in the cells by intein-N and intein-C fusion to produce intact full-length CFTR (without insertions or deletions). Because the inteins only combine in sequence, this approach ensures correct alignment of the CFTR halves, unlike traditional concatemerization approaches.¹⁷ While this approach has previously been proposed by Zhu et al.,^{18,19} to Applicant's knowledge, actual AAVs have not yet been made and tested in CF patient cells. Of note, a dual-AAV approach has recently been proven to be successful for AAV-mediated delivery of a large otoferlin (OTOF) gene to correct hearing loss clinically.²⁰

Disclosed are methods and compositions for the delivery of the Cystic Fibrosis Transmembrane Conductance Regulator (CFTR) gene using intein technology. In aspects, the delivery system employs the coding sequence of a CFTR gene divided into at least two CFTR gene fragments, wherein the at least two fragments total the entirety of the wild-type CFTR gene, and wherein the at least two CFTR gene fragments can be expressed and joined to form a functional CFTR protein. The disclosed compositions comprise a CFTR gene fragment that is attached to (flanked by) an N terminal region or C terminal region of an intein sequence. The CFTR gene can then be delivered in two parts to a cell or tissue, upon which the gene is expressed and the two gene products form a functional CFTR protein. The CFTR fragments can be delivered using two adeno-associated virus (AAV) constructs as described herein or otherwise known in the art. The C-terminal and N-terminal regions of intein bring the two CFTR gene fragments together in vivo to form a functional CFTR protein. Via this process, administration of the intein-CFTR fragment constructs allows for delivery of a fully intact and functional CFTR protein directly to the tissue of the individual, for example via airway epithelial cells.

In aspects, a paired nucleic acid construct system for delivery of a human cystic fibrosis transmembrane conductance regulator (CFTR) protein to an individual in need thereof is disclosed. The system may comprise, for example:
a first construct comprising a first portion (N-terminus) of a human CFTR gene fused to Rhodothernus marinus intein-N cDNA (CFTR-N-inteinN); and
a second construct comprising a second portion (C-terminus) of the human CFTR gene fused to Rhodotherinus marinus intein-C cDNA (inteinC-CFTR-C);
wherein the first portion of the CFTR gene comprises the N terminal portion of the CFTR gene and the second portion of the CFTR gene comprises the carboxy terminal portion of the CFTR gene.

In aspects, the first portion of the CFTR gene encodes for a protein having at least 90%, or at least 95% sequence identity to SEQ ID NO: 6, and the second portion of the CFTR gene encodes for a protein having at least 90%, or at least 95% sequence identity to SEQ ID NO: 8, wherein the first and second protein encoded by the first and second portions of the CFTR gene can be joined to form a complete CFTR protein sequence having wild-type CFTR functionality. That is, the first portion of the CFTR gene and the second portion of the CFTR gene are capable of being fused and expressing a functional CFTR protein.

In aspects, the functional CFTR protein comprises a CFTR R domain. The R domain is defined as amino acids 590-831, encoded by exon 13, which span the region between the C-terminal boundary of the first nucleotide binding fold and the second transmembrane domain, and which is phosphorylated to allow the channel to open.

In aspects, the paired nucleic acid construct system further comprises
a first AAV vector operatively linked to the first construct; and
a second AAV vector operatively linked to the second construct.

In aspects, the first AAV vector and/or the second AAV vector may be the same vector. In aspects, the first AAV vector and/or the second AAV vector may be different AAV vectors. Exemplary AAV vectors are described herein.

In further aspects, a method of using the disclosed paired nucleic acid construct system is disclosed. In one aspect, disclosed is a method of treating an individual having Cystic Fibrosis (CF) comprising administering the paired nucleic acid construct system as described herein, to the individual. The method may comprise contacting the paired nucleic acid construct system with an airway epithelial cell of the individual. In aspects, the administration of the paired nucleic acid construct system provides, restores, or improves CFTR activity in an airway cell of the individual. The individual being treated may be one having a class 1 mutation. For example, the individual may have one more mutations selected from G542X, R553X, and W1282X, with reference to the CFTR gene. In further aspects, the individual may have one or both of a F508 deletion and a 2184delA mutation. In further aspects, the individual may be one who does not respond to a CFTR modulator drug. By “respond” is meant that the individual does not show improvement, or significant improvement, in response to the CFTR modulator drug. In aspects, the CFTR modulator drug is TRIKAFTA (elexacaftor/tezacaftor/ivacaftor).

The paired nucleic acids of the system may be administered via methods described herein. The paired nucleic acids of the system may be administered simultaneously, or sequentially. The paired nucleic acids of the system may be administered every three months, or every six months, or every nine months, or once a year. In aspects, the administration is selected from intranasal delivery, pulmonary delivery, and/or both intranasal and pulmonary delivery.

In further aspects, disclosed is a cell, or plurality of cells that express the paired nucleic acid construct system as described herein.

### Intein Technology

Intein technology has been described. See, e.g., WO2020079034A “Intein proteins and uses thereof,” published Apr. 23, 2020; Chew W L, et al., A multifunctional AAV—CRISPR-Cas9 and its host response. Nat Methods. 2016 October; 13(10):868-74. doi: 10.1038/nmeth.3993. Epub 2016 Sep. 5. PMID: 27595405; PMCID: PMC5374744. (pubmed.ncbi.nlm.nih.gov/27595405/); Yuan et al, “An Intein-Mediated Split-nCas9 system for Base Editing in Plants” ASC Synth. Biol. 2022, 11, 2513-2517; Limberis M P, et al. Transduction efficiencies of novel AAV vectors in mouse airway epithelium in vivo and human ciliated airway epithelium in vitro. Mol Ther. 2009 February; 17(2):294-301. doi: 10.1038/mt.2008.261. Epub 2008 Dec. 9. PMID: 19066597; PMCID: PMC2835069; Lau C H and Suh Y. In vivo genome editing in animals using AAV-CRISPR system: applications to translational research of human disease. F1000Res. 2017 Dec. 20; 6:2153. doi: 10.12688/f1000research.11243.1. PMID: 29333255; PMCID: PMC5749125; Zhu et al, Chin J. Biotech 2010; 26(12): 1710-1718, U.S. Patent Application Publication No. 2023/0090778, “Large gene vectors and delivery and uses thereof,” filed Feb. 5, 2021, U.S. Patent Application Publication No. 2018/0327779, “Multiple vector system and uses thereof,” filed Mar. 3, 2016, and 2004/0077842, “Method of producing biospecific molecules by protein trans-splicing,” filed Nov. 1, 2001.

Inteins are genetic elements transcribed and translated within a host protein from which they self-excise similarly to a protein intron, without leaving amino acid modifications in the final protein product. An intein is a segment of a protein that is able to excise itself and join the remaining portions (the exteins) with a peptide bond in a process known as protein splicing. An N-Intein is an intein fragment located at the N-terminus of (and fused with) a first protein fragment (e.g., a first CFTR fragment) and a C-Intein is an intein fragment located at the C-terminus of (and fused with) the second protein fragment (e.g., a second CFTR fragment), wherein upon expression of the two fragments, the two intein fragments undergo protein trans splicing and are joined to form a full intein, and the two fragments are joined (e.g., the two CFTR fragments), wherein when the two polypeptides form a full length protein, the full length protein is reconstituted (e.g., a full-length CFTR protein, having wild type CFTR functional activity). In aspects, the first intein sequence may be an N-intein sequence and the second intein sequence may be a C-Intein sequence, wherein the N-Intein and the C-Intein may be derived from the same intein gene.

Exemplary inteins include an intein from the DnaE gene (e.g., DNA polymerase III subunit alpha) from cyanobacteria including Nostoc punctiforme (Npu) Synechocystis sp. PCC6803 (Ssp), Fischerella sp. PCC 9605, Scytonema tolypothrichoides, Cyanobacteria bacterium SW_9_47_5, Nodularia spumigena, Nostoc flagelliforme, Crocosphaera watsonii WH 8502, Chroococcidiopsis cubana CCALA 043, Trichodesmium erythraeum. In aspects, the intein is that of Rhodothermus marinus, for example, an intein from Rhodothermus marinus DnaB.^{15,16}

In aspects, the N-intein is that of Rhodothermus marinus DnaB and has a sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 1, wherein SEQ ID NO: 1 is

(SEQ ID NO: 1)
TGTCTGGCTGGCGATACTCTCATTACCCTGGCCGATGGACGACGAGTGCC
TATTAGAGAACTGGTGTCACAGCAGAATTTTTCCGTGTGGGCTCTGAATC
CTCAGACTTACCGCCTGGAGAGGGCTAGAGTGAGTAGAGCTTTCTGTACC
GGCATCAAACCTGTGTACCGCCTCACCACTAGACTGGGGAGATCCATTAG
GGCCACTGCCAACCACCGATTTCTCACACCTCAGGGCTGGAAACGAGTCG
ATGAACTCCAGCCTGGAGATTACCTGGCTCTGCCTAGGAGAATCCCTACT
GCCTCCTGA.

In aspects, the IntN sequence comprises a nucleic acid sequence that encodes for an amino acid sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 2, wherein SEQ ID NO: 2 is

(SEQ ID NO: 2)
CLAGDTLITLADGRRVPIRELVSQQNFSVWALNPQTYRLERARVSRAFCT
GIKPVYRLTTRLGRSIRATANHRFLTPQGWKRVDELQPGDYLALPRRIPT
AS.

In aspects, the Intein C (IntC) is that of Rhodothennus marinus DnaB and has a sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 3, wherein SEQ ID NO: 3 is

(SEQ ID NO: 3)
ATGGCGGCGGCGTGCCCGGAACTGCGTCAGCTGGCGCAGAGCGATGTGTA
TTGGGATCCGATTGTGAGCATTGAACCGGATGGCGTGGAAGAAGTGTTTG
ATCTGACCGTGCCGGGCCCGCATAACTTTGTGGCGAACGATATTATTGCG
CATAACTCT.

In aspects, the Intein C (IntC) sequence comprises a nucleic acid sequence that encodes for an amino acid sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 4, wherein SEQ ID NO: 4 is

(SEQ ID NO: 4)
MAAACPELRQLAQSDVYWDPIVSIEPDGVEEVFDLTVPGPHNFVANDIIA
HNS.

### Cystic Fibrosis Transmembrane Conductance Regulator (CFTR)

The disclosed compositions and methods may be used for delivery of the CFTR protein to a cell, particularly to a cell of an individual having cystic fibrosis (CF). The CFTR gene encodes for the CFTR protein, composed of 1,480 amino acids, which functions as a channel across the membrane of cells. The CFTR coding sequence may be split into two portions. In aspects, the N-intein coding sequence is fused in frame with the sequence coding for the N-terminal portion of the CFTR sequence, and the C-lntein coding sequence is fused in frame with the sequence coding for the C-terminal portion of the CFTR sequence. Upon expression of the two precursor fusion proteins, the inteins undergo autocatalytic excision and form a ligated extein, e.g. the reconstituted CFTR protein.

The disclosed systems and methods employ a paired nucleic acid delivery system. The paired nucleic acid delivery system employs a first CFTR fragment, the first CFTR fragment being capable of expressing a gene product (protein/polypeptide) which can be joined with a second gene product to form a functional CFTR protein. In aspects, the N-terminal portion of the CFTR (CFTR-N) may comprise a sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 5), as follows.

(CFTR-N, SEQ ID NO: 5)
ATGCAGAGGTCGCCTCTGGAAAAGGCCAGCGTTGTCTCCAAACTTTTTTTCAGCTGG
ACCAGACCAATTTTGAGGAAAGGATACAGACAGCGCCTGGAATTGTCAGACATATA
CCAAATCCCTTCTGTTGATTCTGCTGACAATCTATCTGAAAAATTGGAAAGAGAATG
GGATAGAGAGCTGGCTTCAAAGAAAAATCCTAAACTCATTAATGCCCTTCGGCGAT
GTTTTTTCTGGAGATTTATGTTCTATGGAATCTTTTTATATTTAGGGGAAGTCACCAA
AGCAGTACAGCCTCTCTTACTGGGAAGAATCATAGCTTCCTATGACCCGGATAACAA
GGAGGAACGCTCTATCGCGATTTATCTAGGCATAGGCTTATGCCTTCTCTTTATTGTG
AGGACACTGCTCCTACACCCAGCCATTTTTGGCCTTCATCACATTGGAATGCAGATG
AGAATAGCTATGTTTAGTTTGATTTATAAGAAGACTTTAAAGCTGTCAAGCCGTGTT
CTAGATAAAATAAGTATTGGACAACTTGTTAGTCTCCTTTCCAACAACCTGAACAAA
TTTGATGAAGGACTTGCATTGGCACATTTCGTGTGGATCGCTCCTTTGCAAGTGGCA
CTCCTCATGGGGCTAATCTGGGAGTTGTTACAGGCGTCTGCCTTCTGTGGACTTGGTT
TCCTGATAGTCCTTGCCCTTTTTCAGGCTGGGCTAGGGAGAATGATGATGAAGTACA
GAGATCAGAGAGCTGGGAAGATCAGTGAAAGACTTGTGATTACCTCAGAAATGATT
GAAAATATCCAATCTGTTAAGGCATACTGCTGGGAAGAAGCAATGGAAAAAATGAT
TGAAAACTTAAGACAAACAGAACTGAAACTGACTCGGAAGGCAGCCTATGTGAGAT
ACTTCAATAGCTCAGCCTTCTTCTTCTCAGGGTTCTTTGTGGTGTTTTTATCTGTGCTT
CCCTATGCACTAATCAAAGGAATCATCCTCCGGAAAATATTCACCACCATCTCATTC
TGCATTGTTCTGCGCATGGCGGTCACTCGGCAATTTCCCTGGGCTGTACAAACATGG
TATGACTCTCTTGGAGCAATAAACAAAATACAGGATTTCTTACAAAAGCAAGAATAT
AAGACATTGGAATATAACTTAACGACTACAGAAGTAGTGATGGAGAATGTAACAGC
CTTCTGGGAGGAGGGATTTGGGGAATTATTTGAGAAAGCAAAACAAAACAATAACA
ATAGAAAAACTTCTAATGGTGATGACAGCCTCTTCTTCAGTAATTTCTCACTTCTTGG
TACTCCTGTCCTGAAAGATATTAATTTCAAGATAGAAAGAGGACAGTTGTTGGCGGT
TGCTGGATCCACTGGAGCAGGCAAGACTTCACTTCTAATGGTGATTATGGGAGAACT
GGAGCCTTCAGAGGGTAAAATTAAGCACAGTGGAAGAATTTCATTCTGTTCTCAGTT
TTCCTGGATTATGCCTGGCACCATTAAAGAAAATATCATCTTTGGTGTTTCCTATGAT
GAATATAGATACAGAAGCGTCATCAAAGCATGCCAACTAGAAGAGGACATCTCCAA
GTTTGCAGAGAAAGACAATATAGTTCTTGGAGAAGGTGGAATCACACTGAGTGGAG
GTCAACGAGCAAGAATTTCTTTAGCAAGAGCAGTATACAAAGATGCTGATTTGTATT
TATTAGACTCTCCTTTTGGATACCTAGATGTTTTAACAGAAAAAGAAATATTTGAAA
GCTGTGTCTGTAAACTGATGGCTAACAAAACTAGGATTTTGGTCACTTCTAAAATGG
AACATTTAAAGAAAGCTGACAAAATATTAATTTTGCATGAAGGTAGCAGCTATTTTT
ATGGGACATTTTCAGAACTCCAAAATCTACAGCCAGACTTTAGCTCAAAACTCATGG
GATGTGATTCTTTCGACCAATTTAGTGCAGAAAGAAGAAATTCAATCCTAACTGAGA
CCTTACACCGTTTCTCATTAGAAGGAGATGCTCCTGTCTCCTGGACAGAAACAAAAA
AACAATCTTTTAAACAGACTGGAGAGTTTGGGGAAAAAAGGAAGAATTCTATTCTC
AATCCAATCAACTCTATACGAAAATTTTCCATTGTGCAAAAGACTCCCTTACAAATG
AATGGCATCGAAGAGGATTCTGATGAGCCTTTAGAGAGAAGGCTGTCCTTAGTACC
AGATTCTGAGCAGGGAGAGGCGATACTGCCTCGCATCAGCGTGATCAGCACTGGCC
CCACGCTTCAGGCACGAAGGAGGCAGTCTGTCCTGAACCTGATGACACACTCAGTT
AACCAAGGTCAGAACATTCACCGAAAGACAACAGCATCCACACGAAAAGTGTCACT
GGCCCCTCAGGCAAACTTGACTGAACTGGATATATATTCAAGAAGGTTATCTCAAGA
AACTGGCTTGGAAATAAGTGAAGAAATTAACGAAGAAGACTTAAAGGAGTGCTTTT
TTGATGATATGGAG.

In aspects, the CFTR-N sequence comprises a nucleic acid sequence that encodes for an amino acid sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 6, wherein SEQ ID NO: 6 is MQRSPLEKASVVSKLFFSWTRPILRKGYRQRLELSDIYQIPSVDSADNLSEKLEREWDRE LASKKNPKLINALRRCFFWRFMFYGIFLYLGEVTKAVQPLLLGRIIASYDPDNKEERSIAI YLGIGLCLLFIVRTLLLHPAIFGLHHIGMQMRIAMFSLIYKKTLKLSSRVLDKISIGQLVSL LSNNLNKFDEGLALAHFVWIAPLQVALLMGLIWELLQASAFCGLGFLIVLALFQAGLGR MMMKYRDQRAGKISERLVITSEMIENIQSVKAYCWEEAMEKMIENLRQTELKLTRKAA YVRYFNSSAFFFSGFFVVFLSVLPYALIKGIILRKIFTTISFCIVLRMAVTRQFPWAVQTWY DSLGAINKIQDFLQKQEYKTLEYNLTTTEVVMENVTAFWEEGFGELFEKAKQNNNNRK TSNGDDSLFFSNFSLLGTPVLKDINFKIERGQLLAVAGSTGAGKTSLLMVIMGELEPSEG KIKHSGRISFCSQFSWIMPGTIKENIIFGVSYDEYRYRSVIKACQLEEDISKFAEKDNIVLG EGGITLSGGQRARISLARAVYKDADLYLLDSPFGYLDVLTEKEIFESCVCKLMANKTRIL VTSKMEHLKKADKILILHEGSSYFYGTFSELQNLQPDFSSKLMGCDSFDQFSAERRNSIL TETLHRFSLEGDAPVSWTETKKQSFKQTGEFGEKRKNSILNPINSIRKFSIVQKTPLQMNG IEEDSDEPLERRLSLVPDSEQGEAILPRISVISTGPTLQARRRQSVLNLMTHSVNQGQNIH RKTTASTRKVSLAPQANLTELDIYSRRLSQETGLEISEEINEEDLKECFFDDME (SEQ ID NO: 6).

The second CFTR fragment of the paired nucleic acid delivery system is a CFTR fragment capable of expressing a gene product (protein/polypeptide) which can be joined with the first gene product to form a functional CFTR protein. In aspects, the C-terminal portion of the CFTR (CFTR-C) comprises a sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 7, wherein SEQ ID NO: 7 is

(SEQ ID NO: 7)
AGCATACCAGCAGTGACTACATGGAACACATACCTTCGATATATTACTGTCCACAAG
AGCTTAATTTTTGTGCTAATTTGGTGCTTAGTAATTTTTCTGGCAGAGGTGGCTGCTT
CTTTGGTTGTGCTGTGGCTCCTTGGAAACACTCCTCTTCAAGACAAAGGGAATAGTA
CTCATAGTAGAAATAACAGCTATGCAGTGATTATCACCAGCACCAGTTCGTATTATG
TGTTTTACATTTACGTGGGAGTAGCCGACACTTTGCTTGCTATGGGATTCTTCAGAGG
TCTACCACTGGTGCATACTCTAATCACAGTGTCGAAAATTTTACACCACAAAATGTT
ACATTCTGTTCTTCAAGCACCTATGTCAACCCTCAACACGTTGAAAGCAGGTGGGAT
TCTTAATAGATTCTCCAAAGATATAGCAATTTTGGATGACCTTCTGCCTCTTACCATA
TTTGACTTCATCCAGTTGTTATTAATTGTGATTGGAGCTATAGCAGTTGTCGCAGTTT
TACAACCCTACATCTTTGTTGCAACAGTGCCAGTGATAGTGGCTTTTATTATGTTGAG
AGCATATTTCCTCCAAACCTCACAGCAACTCAAACAACTGGAATCTGAAGGCAGGA
GTCCAATTTTCACTCATCTTGTTACAAGCTTAAAAGGACTATGGACACTTCGTGCCTT
CGGACGGCAGCCTTACTTTGAAACTCTGTTCCACAAAGCTCTGAATTTACATACTGC
CAACTGGTTCTTGTACCTGTCAACACTGCGCTGGTTCCAAATGAGAATAGAAATGAT
TTTTGTCATCTTCTTCATTGCTGTTACCTTCATTTCCATTTTAACAACAGGAGAAGGA
GAAGGAAGAGTTGGTATTATCCTGACTTTAGCCATGAATATCATGAGTACATTGCAG
TGGGCTGTAAACTCCAGCATAGATGTGGATAGCTTGATGCGATCTGTGAGCCGAGTC
TTTAAGTTCATTGACATGCCAACAGAAGGTAAACCTACCAAGTCAACCAAACCATAC
AAGAATGGCCAACTCTCGAAAGTTATGATTATTGAGAATTCACACGTGAAGAAAGA
TGACATCTGGCCCTCAGGGGGCCAAATGACTGTCAAAGATCTCACAGCAAAATACA
CAGAAGGTGGAAATGCCATATTAGAGAACATTTCCTTCTCAATAAGTCCTGGCCAGA
GGGTGGGCCTCTTGGGAAGAACTGGATCAGGGAAGAGTACTTTGTTATCAGCTTTTT
TGAGACTACTGAACACTGAAGGAGAAATCCAGATCGATGGTGTGTCTTGGGATTCA
ATAACTTTGCAACAGTGGAGGAAAGCCTTTGGAGTGATACCACAGAAAGTATTTATT
TTTTCTGGAACATTTAGAAAAAACTTGGATCCCTATGAACAGTGGAGTGATCAAGAA
ATATGGAAAGTTGCAGATGAGGTTGGGCTCAGATCTGTGATAGAACAGTTTCCTGGG
AAGCTTGACTTTGTCCTTGTGGATGGGGGCTGTGTCCTAAGCCATGGCCACAAGCAG
TTGATGTGCTTGGCTAGATCTGTTCTCAGTAAGGCGAAGATCTTGCTGCTTGATGAA
CCCAGTGCTCATTTGGATCCAGTAACATACCAAATAATTAGAAGAACTCTAAAACAA
GCATTTGCTGATTGCACAGTAATTCTCTGTGAACACAGGATAGAAGCAATGCTGGAA
TGCCAACAATTTTTGGTCATAGAAGAGAACAAAGTGCGGCAGTACGATTCCATCCA
GAAACTGCTGAACGAGAGGAGCCTCTTCCGGCAAGCCATCAGCCCCTCCGACAGGG
TGAAGCTCTTTCCCCACCGGAACTCAAGCAAGTGCAAGTCTAAGCCCCAGATTGCTG
CTCTGAAAGAGGAGACAGAAGAAGAGGTGCAAGATACAAGGCTT.

In aspects, the CFTR-C sequence comprises a nucleic acid sequence that encodes for an amino acid sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 8, wherein SEQ ID NO: 8 is

(SEQ ID NO: 8)
SIPAVTTWNTYLRYITVHKSLIFVLIWCLVIFLAEVAASLVVLWLLGNTPLQDKGNSTHS
RNNSYAVIITSTSSYYVFYIYVGVADTLLAMGFFRGLPLVHTLITVSKILHHKMLHSVLQ
APMSTLNTLKAGGILNRFSKDIAILDDLLPLTIFDFIQLLLIVIGAIAVVAVLQPYIFVATVP
VIVAFIMLRAYFLQTSQQLKQLESEGRSPIFTHLVTSLKGLWTLRAFGRQPYFETLFHKA
LNLHTANWFLYLSTLRWFQMRIEMIFVIFFIAVTFISILTTGEGEGRVGIILTLAMNIMSTL
QWAVNSSIDVDSLMRSVSRVFKFIDMPTEGKPTKSTKPYKNGQLSKVMIIENSHVKKDD
IWPSGGQMTVKDLTAKYTEGGNAILENISFSISPGQRVGLLGRTGSGKSTLLSAFLRLLNT
EGEIQIDGVSWDSITLQQWRKAFGVIPQKVFIFSGTFRKNLDPYEQWSDQEIWKVADEV
GLRSVIEQFPGKLDFVLVDGGCVLSHGHKQLMCLARSVLSKAKILLLDEPSAHLDPVTY
QIIRRTLKQAFADCTVILCEHRIEAMLECQQFLVIEENKVRQYDSIQKLLNERSLFRQAISP
SDRVKLFPHRNSSKCKSKPQIAALKEETEEEVQDTRL.

### Vector System

A vector system is also described, in which the vector system may be used to express the coding sequence of the first and second CFTR gene fragment in a cell, for example an in vivo cell of a human tissue, for example lung tissue of an individual who has cystic fibrosis (CF). In aspects, the cell is a cell of a human tissue, for example lung tissue of an individual who has CF, and who is not responsive to drug therapy, wherein the drug therapy is the administration of CF modulators (e.g., elexacaftor/tezacaftor/ivacaftor).

The vector system may comprise two vectors, each vector comprising a portion of the CFTR coding sequence flanked by an intein sequence as described above, wherein the 5′ end of the coding sequence is flanked at the 3′ terminus by the sequence of an N-intein, and the 3′ end of the coding sequence of the gene of interest is flanked by the sequence of a C-Intein, such that when both vectors are expressed in a cell, two fusion proteins are produced and the full length CFTR is generated as a result of a spontaneous trans-splicing reaction.

The disclosed vectors and systems concern a virus or virion comprising a polynucleotide, expression construct, or vector construct as described herein. In one aspect, the virus or virion is an AAV virus. Adeno-associated virus (AAV) is widely regarded as a safe and effective method of gene transfer to a variety of tissues. Methods for preparing viruses and virions comprising a heterologous polynucleotide or construct are known in the art. In the case of AAV, cells can be coinfected or transfected with adenovirus or polynucleotide constructs comprising adenovirus genes suitable for AAV helper function. Examples of materials and methods are described, for example, in U.S. Application 20230381342, or U.S. Pat. Nos. 8,137,962, 6,967,018. An AAV virus or AAV vector useful for administration of the paired nucleic acid delivery system can be of any AAV serotype, including, but not limited to, serotype AAV serotype 1 (AAV1), AAV serotype 2 (AAV2), AAV serotype 3 (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), or AAV serotype 12 (AAV12), or any other serotype as known to one of ordinary skill in the viral arts. Features of the vector system may include the AAV backbone, an inverted terminal repeat, a promoter sequence, the CFTR sequence, an intein sequence, and one or more linker sequences as described herein. AAV-mediated delivery of other large-sized genes using inteins has been performed in preclinical models.²⁰⁻²⁴ Both ciliated and non-ciliated airway epithelial cells of different species, including human, mouse and pig, have been shown to be efficiently transduced by apical infection with AAV5, AAV6, AAV6.2, AAV6.2FF and AAV2H22 in vitro and in vivo.^{9, 25-28} Recent single-cell (sc) RNA-seq technology has led to identification of diverse cell types in airway epithelia, including ionocytes, basal, tuft, secretory, goblet and ciliated cells.^{29,30} Ionocytes and secretory cells have been particularly highlighted due to a high-level expression of CFTR in those cells.^{30,31}

In aspects, the AAV vector is AAV6. In further aspects, the AAV vector is AAV6.2, an AAV6 F129L point mutant of AAV6. In yet further aspects, the AAV vector is AAV6.2FF, which is a triple AAV6 mutant containing F129L, Y445F, and Y731F mutations. The AAV6.2FF has improved transduction of lung epithelial cells, including airway epithelial cells, as compared to AAV6. AAV6.2FF is understood by one of ordinary skill in the art and is described in, for example, A Novel Triple-Mutant AAV6 Capsid Induces Rapid and Potent Transgene Expression in the Muscle and Respiratory Tract of Mice, van Lieshout, Laura P. et al., Molecular Therapy Methods & Clinical Development, Volume 9, 323-329 and Thomas, S. P., Spinelli, M. M., Rghei, A. D. et al. Analysis of the impact of pluronic acid on the thermal stability and infectivity of AAV6.2FF. BMC Biotechnol 24, 22 (2024). https://doi.org/10.1186/s12896-024-00853-6.

### Promoters

The constructs described herein may comprise a promoter. Promoters may be ubiquitous, artificial, or tissue specific promoters, including fragments and variants thereof retaining a transcription promoter activity. Exemplary promoters are photoreceptor-specific promoters including photoreceptor-specific human G protein-coupled receptor kinase 1 (GRK1), Interphotoreceptor retinoid binding protein promoter (IRBP), Rhodopsin promoter (RHO), vitelliform macular dystrophy 2 promoter (VMD2), Rhodopsin kinase promoter (RK); Further exemplary promoters are muscle-specific promoters including MCK, MYODI; liver-specific promoters including thyroxine binding globulin (TBG), hybrid liver-specific promoter (HLP); neuron-specific promoters including hSYNl, CaMKIla; kidney-specific promoters including Ksp-cadherinl6, NKCC2. Ubiquitous promoters include ubiquitous cytomegalovirus (CMV) and short CMV promoters Further exemplary promoters include GRK1, TBG, CaMKIla, Ksp-cadherinl6, native gene promoters, cytomegalovirus (CMV) promoter (KF853603.1, bp 149-735), chimeric CMV/chicken beta-actin promoter (CBA) and the truncated form of CBA (smCBA) promoter, Rhodopsin promoter (NG_009115, bp 4205-5010), Interphotoreceptor retinoid binding protein promoter (NG_029718.1, bp 4777-5011), vitelliform macular dystrophy 2 promoter (NG_009033.1, bp 4870-5470), PR-specific human G protein-coupled receptor kinase 1 (hGRKI; AY327580.1 bp1793-2087 or bp 1793-1991) (Haire et al. 2006; U.S. Pat. No. 8,298,818). However, any suitable promoter known in the art may be used.

In aspects, the promoter is the SMVP promoter. The SMVP promoter is generated by fusing the SV40 enhancer-CMV-promoter-chimeric intron. In aspects, the SMVP promoter sequence may comprise a sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 9, wherein SEQ ID NO: 9 is

(SEQ ID NO: 9)
GGCATTGATTATTGACTAGTTATTAATAGTAATCAATTACGGGGTCATTAGTTCATA
GCCCATATATGGAGTTCCGCGTTACATAACTTACGGTAAATGGCCCGCCTGGCTGAC
CGCCCAACGACCCCCGCCCATTGACGTCAATAATGACGTATGTTCCCATAGTAACGC
CAATAGGGACTTTCCATTGACGTCAATGGGTGGAGTATTTACGGTAAACTGCCCACT
TGGCAGTACATCAAGTGTATCATATGCCAAGTCCGCCCCCTATTGACGTCAATGACG
GTAAATGGCCCGCCTGGCATTATGCCCAGTACATGACCTTACGGGACTTTCCTACTT
GGCAGTACATCTACGTATTAGTCATCGCTATTACCATGGTGATGCGGTTTTGGCAGT
ACACCAATGGGCGTGGATAGCGGTTTGACTCACGGGGATTTCCAAGTCTCCACCCCA
TTGACGTCAATGGGAGTTTGTTTTGGCACCAAAATCAACGGGACTTTCCAAAATGTC
GTAATAACCCCGCCCCGTTGACGCAAATGGGCGGTAGGCGTGTACGGTGGGAGGTC
TATATAAGCAGAGGTCGTTTAGTGAACCGTCAGATCACTAGTAGCTTTATTGCGGTA
GTTTATCACAGTTAAATTGCTAACGCAGTCAGTGCTCGACTGATCACAGGTAAGTAT
CAAGGTTACAAGACAGGTTTAAGGAGGCCAATAGAAACTGGGCTTGTCGAGACAGA
GAAGATTCTTGCGTTTCTGATAGGCACCTATTGGTCTTACTGACATCCACTTTGCCTT
TCTCTCCACAG.

Promoters can be incorporated into a construct using standard techniques known in the art. Multiple copies of promoters or multiple promoters can be used in a vector of the invention. In aspects, the promoter can be positioned about the same distance from the transcription start site as it is from the transcription start site in its natural genetic environment. Some variation in this distance is permitted without substantial decrease in promoter activity. A transcription start site is typically included in the 5′ construct but not in the 3′ construct. In further aspects, a transcription start site may be included in the 3′ construct upstream of the degradation signal.

### Sequence Variation

As those skilled in the art can readily appreciate, there can be a number of variant sequences of a protein found in nature, in addition to those variants that can be artificially created by the skilled artisan in the lab. The polynucleotides and polypeptides of the subject invention encompasses those specifically exemplified herein, as well as any natural variants thereof, as well as any variants which can be created artificially, so long as those variants retain the desired functional activity. Also within the scope of the subject invention are polypeptides which have the same amino acid sequences of a polypeptide exemplified herein except for amino acid substitutions, additions, or deletions within the sequence of the polypeptide, as long as these variant polypeptides retain substantially the same relevant functional activity as the polypeptides specifically exemplified herein. For example, conservative amino acid substitutions within a polypeptide which do not affect the function of the polypeptide would be within the scope of the subject invention. Thus, the polypeptides disclosed herein should be understood to include variants and fragments, as discussed above, of the specifically exemplified sequences. The subject invention further includes nucleotide sequences which encode the polypeptides disclosed herein. These nucleotide sequences can be readily constructed by those skilled in the art having the knowledge of the protein and amino acid sequences which are presented herein. As would be appreciated by one skilled in the art, the degeneracy of the genetic code enables the artisan to construct a variety of nucleotide sequences that encode a particular polypeptide or protein. The choice of a particular nucleotide sequence could depend, for example, upon the codon usage of a particular expression system or host cell. Polypeptides having substitution of amino acids other than those specifically exemplified in the subject polypeptides are also contemplated within the scope of the present invention. For example, non-natural amino acids can be substituted for the amino acids of a polypeptide of the invention, so long as the polypeptide having substituted amino acids retains substantially the same activity as the polypeptide in which amino acids have not been substituted. Examples of non-natural amino acids include, but are not limited to, ornithine, citrulline, hydroxyproline, homoserine, phenylglycine, taurine, iodotyrosine, 2,4-diaminobutyric acid, a-amino isobutyric acid, 4-aminobutyric acid, 2-amino butyric acid, γ-amino butyric acid, ε-amino hexanoic acid, 6-amino hexanoic acid, 2-amino isobutyiic acid, 3-amino propionic acid, norleucine, norvaline, sarcosine, homocitrulline, cysteic acid, τ-butylglycine, τ-butylalanine, phenylglycine, cyclohexylalanine, β-alanine, fluoro-amino acids, designer amino acids such as β-methyl amino acids, C-methyl amino acids, N-methyl amino acids, and amino acid analogues in general. Non-natural amino acids also include amino acids having derivatized side groups. Furthermore, any of the amino acids in the protein can be of the D (dextrorotary) form or L (levorotary) form. Amino acids can be generally categorized in the following classes: non-polar, uncharged polar, basic, and acidic. Conservative substitutions whereby a polypeptide having an amino acid of one class is replaced with another amino acid of the same class fall within the scope of the subject invention so long as the polypeptide having the substitution still retains substantially the same biological activity as a polypeptide that does not have the substitution. Table 1 provides a listing of examples of amino acids belonging to each class.

TABLE 1

| Class of Amino Acid | Examples of Amino Acids |
|---|---|
| Nonpolar | Ala, Val, Leu, Ile, Pro, Met, |
| | Phe, Trp |
| Unchanged Polar | Gly, Ser, Thr, Cys, Try, Asn, |
| | Gln |
| Acidic | Asp, Glu |
| Basic | Lys, Arg, His |

Also within the scope of the subject invention are polynucleotides which have the same nucleotide sequences of a polynucleotide exemplified herein except for nucleotide substitutions, additions, or deletions within the sequence of the polynucleotide, as long as these variant polynucleotides retain substantially the same relevant functional activity as the polynucleotides specifically exemplified herein (e.g., they encode a protein having the same amino acid sequence or the same functional activity as encoded by the exemplified polynucleotide). Thus, the polynucleotides disclosed herein should be understood to include variants and fragments, as discussed above, of the specifically exemplified sequences.

The subject invention also contemplates those polynucleotide molecules having sequences which are sufficiently homologous with the polynucleotide sequences of the invention so as to permit hybridization with that sequence under standard stringent conditions and standard methods (Maniatis, T. et al, 1982). Polynucleotides described herein can also be defined in terms of more particular identity and/or similarity ranges with those exemplified herein. The sequence identity will typically be greater than 60%, preferably greater than 75%, more preferably greater than 80%, even more preferably greater than 90%, and can be greater than 95%. The identity and/or similarity of a sequence can be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% or greater as compared to a sequence exemplified herein. Unless otherwise specified, as used herein percent sequence identity and/or similarity of two sequences can be determined using the algorithm of Karlin and Altschul (1990), modified as in Karlin and Altschul (1993). Such an algorithm is incorporated into the NBLAST and XBLAST programs of Altschul et al. (1990). BLAST searches can be performed with the NBLAST program, score=100, wordlength=12, to obtain sequences with the desired percent sequence identity. To obtain gapped alignments for comparison purposes, Gapped BLAST can be used as described in Altschul et al. (1997). When utilizing BLAST and Gapped BLAST programs, the default parameters of the respective programs (NBLAST and XBLAST) can be used. See NCBI/NIH website.

### Additional Features

In aspects, the first vector and/or the second vector further comprise a 5′-terminal repeat (5′-TR) nucleotide sequence and a 3′-terminal repeat (3′-TR) nucleotide sequence, preferably the 5′-TR is a 5′-inverted terminal repeat (5′-ITR) nucleotide sequence and the 3′-TR is a 3′-inverted terminal repeat (3′-ITR) nucleotide sequence.

In aspects, the first vector and/or the second vector further comprise a poly-adenylation signal nucleotide sequence.

In aspects, the coding sequence is split into the first portion and the second portion at a position consisting of a nucleophile amino acid which does not fall within a structural domain or a functional domain of the encoded protein product, wherein the nucleophile amino acid is selected from serine, threonine, or cysteine.

In aspects, at least one of the first vector and the second vector further comprise at least one enhancer or regulatory nucleotide sequence, operably linked to the coding sequence.

### Flag-CFTR-N-intN Construct

In aspects of the dual nucleic acid delivery system, a first construct comprising a first CFTR sequence and a first intein is disclosed. An exemplary first construct is the “Flag-CFTR-N-intN” construct. It should be noted, however, that the “flag” portion is optional and may be omitted for clinical administration to an individual. The Flag-CFTR-N-intN for use in the disclosed compositions and methods may be made by employing routine molecular biology techniques as understood in the art and as described in Chew W L, Tabebordbar M, Cheng J K, et al. A multifunctional AAV-CRISPR-Cas9 and its host response. Nat Methods. 2016; 13(10):868-874. doi:10.1038/nmeth.3993. In brief, the human CFTR N-terminal lobe may be fused with the Rhodotherinus marinus N-split-intein and the human CFTR C-terminal lobe with the Rhodothermus marinus C-split-intein. The component sequences for manufacture of the Flag-CFTR-N-intN construct and delivery vehicle are described below.

The Flag-CFTR-N-intN construct may comprise a tag. The tag may be a flag sequence. The flag sequence can serve as a marker for the detection of the CFTR protein following its delivery to a cell or an individual. In aspects, the flag component is included for experimental applications. In alternative aspects, the flag component is incorporated for therapeutic applications. In aspects where the application is clinical, the construct is designed without the inclusion of a flag. In scenarios where a flag is used, one example of a suitable flag sequence is a sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 10, wherein SEQ ID NO: 10 is

(SEQ ID NO: 10)
ATGGACTACAAAGACCATGACGGTGATTATAAAGATCATGACATCGATTA
CAAGGATGACGATGACAAGCTT.

In aspects, the Flag sequence comprises a nucleic acid sequence that encodes for an amino acid sequence having at least 90%, or at least 91%, or at least 92%, or at least 93%, or at least 94%, or at least 95%, or at least 96%, or at least 97%, or at least 98%, or at least 99%, or 100% sequence identity to

### MDYKDHDGDYKDHDIDYKDDDDKL (Flag, SEQ ID NO: 11).

The human CFTR-N sequence of the construct comprises a sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 5, wherein SEQ ID NO: 5 is

(CFTR-N, SEQ ID NO: 5)
ATGCAGAGGTCGCCTCTGGAAAAGGCCAGCGTTGTCTCCAAACTTTTTTTCAGCTGG
ACCAGACCAATTTTGAGGAAAGGATACAGACAGCGCCTGGAATTGTCAGACATATA
CCAAATCCCTTCTGTTGATTCTGCTGACAATCTATCTGAAAAATTGGAAAGAGAATG
GGATAGAGAGCTGGCTTCAAAGAAAAATCCTAAACTCATTAATGCCCTTCGGCGAT
GTTTTTTCTGGAGATTTATGTTCTATGGAATCTTTTTATATTTAGGGGAAGTCACCAA
AGCAGTACAGCCTCTCTTACTGGGAAGAATCATAGCTTCCTATGACCCGGATAACAA
GGAGGAACGCTCTATCGCGATTTATCTAGGCATAGGCTTATGCCTTCTCTTTATTGTG
AGGACACTGCTCCTACACCCAGCCATTTTTGGCCTTCATCACATTGGAATGCAGATG
AGAATAGCTATGTTTAGTTTGATTTATAAGAAGACTTTAAAGCTGTCAAGCCGTGTT
CTAGATAAAATAAGTATTGGACAACTTGTTAGTCTCCTTTCCAACAACCTGAACAAA
TTTGATGAAGGACTTGCATTGGCACATTTCGTGTGGATCGCTCCTTTGCAAGTGGCA
CTCCTCATGGGGCTAATCTGGGAGTTGTTACAGGCGTCTGCCTTCTGTGGACTTGGTT
TCCTGATAGTCCTTGCCCTTTTTCAGGCTGGGCTAGGGAGAATGATGATGAAGTACA
GAGATCAGAGAGCTGGGAAGATCAGTGAAAGACTTGTGATTACCTCAGAAATGATT
GAAAATATCCAATCTGTTAAGGCATACTGCTGGGAAGAAGCAATGGAAAAAATGAT
TGAAAACTTAAGACAAACAGAACTGAAACTGACTCGGAAGGCAGCCTATGTGAGAT
ACTTCAATAGCTCAGCCTTCTTCTTCTCAGGGTTCTTTGTGGTGTTTTTATCTGTGCTT
CCCTATGCACTAATCAAAGGAATCATCCTCCGGAAAATATTCACCACCATCTCATTC
TGCATTGTTCTGCGCATGGCGGTCACTCGGCAATTTCCCTGGGCTGTACAAACATGG
TATGACTCTCTTGGAGCAATAAACAAAATACAGGATTTCTTACAAAAGCAAGAATAT
AAGACATTGGAATATAACTTAACGACTACAGAAGTAGTGATGGAGAATGTAACAGC
CTTCTGGGAGGAGGGATTTGGGGAATTATTTGAGAAAGCAAAACAAAACAATAACA
ATAGAAAAACTTCTAATGGTGATGACAGCCTCTTCTTCAGTAATTTCTCACTTCTTGG
TACTCCTGTCCTGAAAGATATTAATTTCAAGATAGAAAGAGGACAGTTGTTGGCGGT
TGCTGGATCCACTGGAGCAGGCAAGACTTCACTTCTAATGGTGATTATGGGAGAACT
GGAGCCTTCAGAGGGTAAAATTAAGCACAGTGGAAGAATTTCATTCTGTTCTCAGTT
TTCCTGGATTATGCCTGGCACCATTAAAGAAAATATCATCTTTGGTGTTTCCTATGAT
GAATATAGATACAGAAGCGTCATCAAAGCATGCCAACTAGAAGAGGACATCTCCAA
GTTTGCAGAGAAAGACAATATAGTTCTTGGAGAAGGTGGAATCACACTGAGTGGAG
GTCAACGAGCAAGAATTTCTTTAGCAAGAGCAGTATACAAAGATGCTGATTTGTATT
TATTAGACTCTCCTTTTGGATACCTAGATGTTTTAACAGAAAAAGAAATATTTGAAA
GCTGTGTCTGTAAACTGATGGCTAACAAAACTAGGATTTTGGTCACTTCTAAAATGG
AACATTTAAAGAAAGCTGACAAAATATTAATTTTGCATGAAGGTAGCAGCTATTTTT
ATGGGACATTTTCAGAACTCCAAAATCTACAGCCAGACTTTAGCTCAAAACTCATGG
GATGTGATTCTTTCGACCAATTTAGTGCAGAAAGAAGAAATTCAATCCTAACTGAGA
CCTTACACCGTTTCTCATTAGAAGGAGATGCTCCTGTCTCCTGGACAGAAACAAAAA
AACAATCTTTTAAACAGACTGGAGAGTTTGGGGAAAAAAGGAAGAATTCTATTCTC
AATCCAATCAACTCTATACGAAAATTTTCCATTGTGCAAAAGACTCCCTTACAAATG
AATGGCATCGAAGAGGATTCTGATGAGCCTTTAGAGAGAAGGCTGTCCTTAGTACC
AGATTCTGAGCAGGGAGAGGCGATACTGCCTCGCATCAGCGTGATCAGCACTGGCC
CCACGCTTCAGGCACGAAGGAGGCAGTCTGTCCTGAACCTGATGACACACTCAGTT
AACCAAGGTCAGAACATTCACCGAAAGACAACAGCATCCACACGAAAAGTGTCACT
GGCCCCTCAGGCAAACTTGACTGAACTGGATATATATTCAAGAAGGTTATCTCAAGA
AACTGGCTTGGAAATAAGTGAAGAAATTAACGAAGAAGACTTAAAGGAGTGCTTTT
TTGATGATATGGAG.

In aspects, the CFTR-N sequence comprises a nucleic acid sequence that encodes for an amino acid sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 6, wherein SEQ ID NO: 6 is

(SEQ ID NO: 6)
MQRSPLEKASVVSKLFFSWTRPILRKGYRQRLELSDIYQIPSVDSADNLSEKLEREWDRE
LASKKNPKLINALRRCFFWRFMFYGIFLYLGEVTKAVQPLLLGRIIASYDPDNKEERSIAI
YLGIGLCLLFIVRTLLLHPAIFGLHHIGMQMRIAMFSLIYKKTLKLSSRVLDKISIGQLVSL
LSNNLNKFDEGLALAHFVWIAPLQVALLMGLIWELLQASAFCGLGFLIVLALFQAGLGR
MMMKYRDQRAGKISERLVITSEMIENIQSVKAYCWEEAMEKMIENLRQTELKLTRKAA
YVRYFNSSAFFFSGFFVVFLSVLPYALIKGIILRKIFTTISFCIVLRMAVTRQFPWAVQTWY
DSLGAINKIQDFLQKQEYKTLEYNLTTTEVVMENVTAFWEEGFGELFEKAKQNNNNRK
TSNGDDSLFFSNFSLLGTPVLKDINFKIERGQLLAVAGSTGAGKTSLLMVIMGELEPSEG
KIKHSGRISFCSQFSWIMPGTIKENIIFGVSYDEYRYRSVIKACQLEEDISKFAEKDNIVLG
EGGITLSGGQRARISLARAVYKDADLYLLDSPFGYLDVLTEKEIFESCVCKLMANKTRIL
VTSKMEHLKKADKILILHEGSSYFYGTFSELQNLQPDFSSKLMGCDSFDQFSAERRNSIL
TETLHRFSLEGDAPVSWTETKKQSFKQTGEFGEKRKNSILNPINSIRKFSIVQKTPLQMNG
IEEDSDEPLERRLSLVPDSEQGEAILPRISVISTGPTLQARRRQSVLNLMTHSVNQGQNIH
RKTTASTRKVSLAPQANLTELDIYSRRLSQETGLEISEEINEEDLKECFFDDME.

In aspects, the Rhodothermus marinus Intein-N (intN) sequence is a sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 1, wherein SEQ ID NO: 1 is

(SEQ ID NO: 1)
TGTCTGGCTGGCGATACTCTCATTACCCTGGCCGATGGACGACGAGTGCC
TATTAGAGAACTGGTGTCACAGCAGAATTTTTCCGTGTGGGCTCTGAATC
CTCAGACTTACCGCCTGGAGAGGGCTAGAGTGAGTAGAGCTTTCTGTACC
GGCATCAAACCTGTGTACCGCCTCACCACTAGACTGGGGAGATCCATTAG
GGCCACTGCCAACCACCGATTTCTCACACCTCAGGGCTGGAAACGAGTCG
ATGAACTCCAGCCTGGAGATTACCTGGCTCTGCCTAGGAGAATCCCTACT
GCCTCCTGA.

In aspects, the IntN sequence comprises a nucleic acid sequence that encodes for an amino acid sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 2, wherein SEQ ID NO: 2 is

(SEQ ID NO: 2)
CLAGDTLITLADGRRVPIRELVSQQNFSVWALNPQTYRLERARVSRAFCT
GIKPVYRLTTRLGRSIRATANHRFLTPQGWKRVDELQPGDYLALPRRIPT
AS

Any of the above sequences may be inserted into an AAV vector for delivery to a cell or an individual. In aspects, the sequences are operatively linked. In aspects, each of the sequences are operatively linked via a linker sequence as described herein. In aspects, the CFTR fragment and intein are directly connected, and are not connected via a linker sequence. In aspects, the CFTR-N and Int-N are directly connected, and are not connected via a linker sequence. In aspects, the CFTR-C and Int-C are directly connected, and are not connected via a linker sequence. In aspects, the above sequences may be inserted into an AAV vector for delivery. In aspects, the full sequence of the Flag-CFTR-N-intN is a sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 12, wherein SEQ ID NO: 12 is

(SEQ ID NO: 12)
ATG GAC TAC AAA GAC CAT GAC GGT GAT TAT AAA GAT CAT GAC ATC GAT TAC
AAG GAT GAC GAT GAC AAG CTT ATG CAG AGG TCG CCT CTG GAA AAG GCC
AGC GTT GTC TCC AAA CTT TTT TTC AGC TGG ACC AGA CCA ATT TTG AGG AAA
GGA TAC AGA CAG CGC CTG GAA TTG TCA GAC ATA TAC CAA ATC CCT TCT GTT
GAT TCT GCT GAC AAT CTA TCT GAA AAA TTG GAA AGA GAA TGG GAT AGA
GAG CTG GCT TCA AAG AAA AAT CCT AAA CTC ATT AAT GCC CTT CGG CGA TGT
TTT TTC TGG AGA TTT ATG TTC TAT GGA ATC TTT TTA TAT TTA GGG GAA GTC
ACC AAA GCA GTA CAG CCT CTC TTA CTG GGA AGA ATC ATA GCT TCC TAT GAC
CCG GAT AAC AAG GAG GAA CGC TCT ATC GCG ATT TAT CTA GGC ATA GGC TTA
TGC CTT CTC TTT ATT GTG AGG ACA CTG CTC CTA CAC CCA GCC ATT TTT GGC
CTT CAT CAC ATT GGA ATG CAG ATG AGA ATA GCT ATG TTT AGT TTG ATT TAT
AAG AAG ACT TTA AAG CTG TCA AGC CGT GTT CTA GAT AAA ATA AGT ATT GGA
CAA CTT GTT AGT CTC CTT TCC AAC AAC CTG AAC AAA TTT GAT GAA GGA CTT
GCA TTG GCA CAT TTC GTG TGG ATC GCT CCT TTG CAA GTG GCA CTC CTC ATG
GGG CTA ATC TGG GAG TTG TTA CAG GCG TCT GCC TTC TGT GGA CTT GGT TTC
CTG ATA GTC CTT GCC CTT TTT CAG GCT GGG CTA GGG AGA ATG ATG ATG AAG
TAC AGA GAT CAG AGA GCT GGG AAG ATC AGT GAA AGA CTT GTG ATT ACC
TCA GAA ATG ATT GAA AAT ATC CAA TCT GTT AAG GCA TAC TGC TGG GAA GAA
GCA ATG GAA AAA ATG ATT GAA AAC TTA AGA CAA ACA GAA CTG AAA CTG
ACT CGG AAG GCA GCC TAT GTG AGA TAC TTC AAT AGC TCA GCC TTC TTC TTC
TCA GGG TTC TTT GTG GTG TTT TTA TCT GTG CTT CCC TAT GCA CTA ATC AAA
GGA ATC ATC CTC CGG AAA ATA TTC ACC ACC ATC TCA TTC TGC ATT GTT CTG
CGC ATG GCG GTC ACT CGG CAA TTT CCC TGG GCT GTA CAA ACA TGG TAT GAC
TCT CTT GGA GCA ATA AAC AAA ATA CAG GAT TTC TTA CAA AAG CAA GAA TAT
AAG ACA TTG GAA TAT AAC TTA ACG ACT ACA GAA GTA GTG ATG GAG AAT
GTA ACA GCC TTC TGG GAG GAG GGA TTT GGG GAA TTA TTT GAG AAA GCA
AAA CAA AAC AAT AAC AAT AGA AAA ACT TCT AAT GGT GAT GAC AGC CTC
TTC TTC AGT AAT TTC TCA CTT CTT GGT ACT CCT GTC CTG AAA GAT ATT AAT
TTC AAG ATA GAA AGA GGA CAG TTG TTG GCG GTT GCT GGA TCC ACT GGA
GCA GGC AAG ACT TCA CTT CTA ATG GTG ATT ATG GGA GAA CTG GAG CCT TCA
GAG GGT AAA ATT AAG CAC AGT GGA AGA ATT TCA TTC TGT TCT CAG TTT TCC
TGG ATT ATG CCT GGC ACC ATT AAA GAA AAT ATC ATC TTT GGT GTT TCC TAT
GAT GAA TAT AGA TAC AGA AGC GTC ATC AAA GCA TGC CAA CTA GAA GAG
GAC ATC TCC AAG TTT GCA GAG AAA GAC AAT ATA GTT CTT GGA GAA GGT
GGA ATC ACA CTG AGT GGA GGT CAA CGA GCA AGA ATT TCT TTA GCA AGA
GCA GTA TAC AAA GAT GCT GAT TTG TAT TTA TTA GAC TCT CCT TTT GGA TAC
CTA GAT GTT TTA ACA GAA AAA GAA ATA TTT GAA AGC TGT GTC TGT AAA CTG
ATG GCT AAC AAA ACT AGG ATT TTG GTC ACT TCT AAA ATG GAA CAT TTA AAG
AAA GCT GAC AAA ATA TTA ATT TTG CAT GAA GGT AGC AGC TAT TTT TAT GGG
ACA TTT TCA GAA CTC CAA AAT CTA CAG CCA GAC TTT AGC TCA AAA CTC ATG
GGA TGT GAT TCT TTC GAC CAA TTT AGT GCA GAA AGA AGA AAT TCA ATC CTA
ACT GAG ACC TTA CAC CGT TTC TCA TTA GAA GGA GAT GCT CCT GTC TCC TGG
ACA GAA ACA AAA AAA CAA TCT TTT AAA CAG ACT GGA GAG TTT GGG GAA
AAA AGG AAG AAT TCT ATT CTC AAT CCA ATC AAC TCT ATA CGA AAA TTT TCC
ATT GTG CAA AAG ACT CCC TTA CAA ATG AAT GGC ATC GAA GAG GAT TCT GAT
GAG CCT TTA GAG AGA AGG CTG TCC TTA GTA CCA GAT TCT GAG CAG GGA
GAG GCG ATA CTG CCT CGC ATC AGC GTG ATC AGC ACT GGC CCC ACG CTT CAG
GCA CGA AGG AGG CAG TCT GTC CTG AAC CTG ATG ACA CAC TCA GTT AAC
CAA GGT CAG AAC ATT CAC CGA AAG ACA ACA GCA TCC ACA CGA AAA GTG
TCA CTG GCC CCT CAG GCA AAC TTG ACT GAA CTG GAT ATA TAT TCA AGA AGG
TTA TCT CAA GAA ACT GGC TTG GAA ATA AGT GAA GAA ATT AAC GAA GAA
GAC TTA AAG GAG TGC TTT TTT GAT GAT ATG GAG TGT CTG GCT GGC GAT ACT
CTC ATT ACC CTG GCC GAT GGA CGA CGA GTG CCT ATT AGA GAA CTG GTG TCA
CAG CAG AAT TTT TCC GTG TGG GCT CTG AAT CCT CAG ACT TAC CGC CTG GAG
AGG GCT AGA GTG AGT AGA GCT TTC TGT ACC GGC ATC AAA CCT GTG TAC CGC
CTC ACC ACT AGA CTG GGG AGA TCC ATT AGG GCC ACT GCC AAC CAC CGA TTT
CTC ACA CCT CAG GGC TGG AAA CGA GTC GAT GAA CTC CAG CCT GGA GAT
TAC CTG GCT CTG CCT AGG AGA ATC CCT ACT GCC TCC TGA.

In aspects, the full sequence of the Flag-CFTR-N-intN expresses a protein having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 13, wherein SEQ ID NO: 13 is

(SEQ ID NO: 13)
M D Y K D H D G D Y K D H D I D Y K D D D D K L M
Q R S P L E K A S V V S K L F F S W T R P I L R K
G Y R Q R L E L S D I Y Q I P S V D S A D N L S E
K L E R E W D R E L A S K K N P K L I N A L R R C
F F W R F M F Y G I F L Y L G E V T K A V Q P L L
L G R I I A S Y D P D N K E E R S I A I Y L G I G
L C L L F I V R T L L L H P A I F G L H H I G M Q
M R I A M F S L I Y K K T L K L S S R V L D K I S
I G Q L V S L L S N N L N K F D E G L A L A H F V
W I A P L Q V A L L M G L I W E L L Q A S A F C G
L G F L I V L A L F Q A G L G R M M M K Y R D Q R
A G K I S E R L V I T S E M I E N I Q S V K A Y C
W E E A M E K M I E N L R Q T E L K L T R K A A Y
V R Y F N S S A F F F S G F F V V F L S V L P Y A
L I K G I I L R K I F T T I S F C I V L R M A V T
R Q F P W A V Q T W Y D S L G A I N K I Q D F L Q
K Q E Y K T L E Y N L T T T E V V M E N V T A F W
E E G F G E L F E K A K Q N N N N R K T S N G D D
S L F F S N F S L L G T P V L K D I N F K I E R G
Q L L A V A G S T G A G K T S L L M V I M G E L E
P S E G K I K H S G R I S F C S Q F S W I M P G T
I K E N I I F G V S Y D E Y R Y R S V I K A C Q L
E E D I S K F A E K D N I V L G E G G I T L S G G
Q R A R I S L A R A V Y K D A D L Y L L D S P F G
Y L D V L T E K E I F E S C V C K L M A N K T R I
L V T S K M E H L K K A D K I L I L H E G S S Y F
Y G T F S E L Q N L Q P D F S S K L M G C D S F D
Q F S A E R R N S I L T E T L H R F S L E G D A P
V S W T E T K K Q S F K Q T G E F G E K R K N S I
L N P I N S I R K F S I V Q K T P L Q M N G I E E
D S D E P L E R R L S L V P D S E Q G E A I L P R
I S V I S T G P T L Q A R R R Q S V L N L M T H S
V N Q G Q N I H R K T T A S T R K V S L A P Q A N
L T E L D I Y S R R L S Q E T G L E I S E E I N E
E D L K E C F F D D M E C L A G D T L I T L A D G
R R V P I R E L V S Q Q N F S V W A L N P Q T Y R
L E R A R V S R A F C T G I K P V Y R L T T R L G
R S I R A T A N H R F L T P Q G W K R V D E L Q P
G D Y L A L P R R I P T A S.

The pAAV-SMVP-Flag-CFTR-N-intN nucleic acid construct may be provided in an expression construct, for example, for amplification in bacteria. An exemplary expression construct may comprise an ORI sequence, a linker sequence, an AAV2ITR sequence, and a second linker sequence. For example, in aspects, the construct may comprise an ORI sequence, the sequence having at least 90%, or at least 91%, or at least 92%, or at least 93%, or at least 94%, or at least 95%, or at least 96%, or at least 97%, or at least 98%, or at least 99%, or 100% sequence identity to

(ORI sequence, SEQ ID NO: 14)
ACGCTTCCCGAAGGGAGAAAGGCGGACAGGTATCCGGTAAGCGGCAGGGT
CGGAACAGGAGAGCGCACGAGGGAGCTTCCAGGGGGAAACGCCTGGTATC
TTTATAGTCCTGTCGGGTTTCGCCACCTCTGACTTGAGCGTCGATTTTTG
TGATGCTCGTCAGGGGGGCGGAGCCTATGGAA.

In aspects, the pAAV-SMVP-Flag-CFTR-N-intN may comprise a first linker sequence. The first linker sequence may be used to link (operatively connect) the ORI sequence above and the AAV2 ITR sequence. In this aspect, the sequence may comprise a sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 15, wherein SEQ ID NO: 15 is

(SEQ ID NO: 15)
AAACGCCAGCAACGCGGCCTTTTTACGGTTCCTGGCCTTTTGCTGCGGTT
TTGCTCACATGTTCTTTCCTGCGTTATCCCCTGATTCTGTGGATAACCGT
ATTACCGCCTTTGAGTGAGCTGATACCGCTCGCCGCAGCCGAACGACCGA
GCGCAGCGAGTCAGTGAGCGAGGAAGCGGAAGAGCGCCCAATACGCAAAC
CGCCTCTCCCCGCGCGTTGGCCGATTCATTAATGCAGCTGGCACGACAGG
TTTCCCGACTGGAAAGCGGGCAGTGAGCGCAACGCAATTAATGTGAGTTA
GCTCACTCATTAGGCACCCCAGGCTTTACACTTTATGCTTCCGGCTCGTA
TGTTGTGTGGAATTGTGAGCGGATAACAATTTCACACAGGAAACAGCTAT
GACCATGATTACGCCAGATTTAATTAAGG.

In aspects, the pAAV-SMVP-Flag-CFTR-N-intN may comprise an AAV2 ITR sequence. The AAV2 ITR sequence may comprise a sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 16, wherein SEQ ID NO: 16 is

(SEQ ID NO: 16)
CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGCCAACTCCATCACTAGGGGTTCCT.

In aspects, the pAAV-SMVP-Flag-CFTR-N-intN may comprise a second linker sequence. In this aspect, the second linker sequence may comprise a sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 17, wherein SEQ ID NO: 17 is

(SEQ ID NO: 17)
TGTAGTTAATGATTAACCCGCCATGCTACTTATCTACCTGCGGCCGCGGA
ATGTGTGTCAGTTAGGGTGTGGAAAGTCCCCAGGCTCCCCAGCAGGCAGA
AGTATGCAAAGCATGCATCTCAATTAGTCAGCAACCAGTCCCGGTCTCCT
CCCATGCATGTCAATATTGGCCATTAGCCATATTATTCATTGGTTATATA
GCATAAATCAATATTGGCTATTGGCCATTGCATACGTTGTATCTATATCA
TAATATGTACATTTATATTGGCTCATGTCCAATATGACCGCCATGTT.

In aspects, the pAAV-SMVP-Flag-CFTR-N-intN may comprise a SMPV promoter sequence. In this aspect, the SMPV promoter sequence may comprise a sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 9, wherein SEQ ID NO: 9 is

(SEQ ID NO: 9)
GGCATTGATTATTGACTAGTTATTAATAGTAATCAATTACGGGGTCATTA
GTTCATAGCCCATATATGGAGTTCCGCGTTACATAACTTACGGTAAATGG
CCCGCCTGGCTGACCGCCCAACGACCCCCGCCCATTGACGTCAATAATGA
CGTATGTTCCCATAGTAACGCCAATAGGGACTTTCCATTGACGTCAATGG
GTGGAGTATTTACGGTAAACTGCCCACTTGGCAGTACATCAAGTGTATCA
TATGCCAAGTCCGCCCCCTATTGACGTCAATGACGGTAAATGGCCCGCCT
GGCATTATGCCCAGTACATGACCTTACGGGACTTTCCTACTTGGCAGTAC
ATCTACGTATTAGTCATCGCTATTACCATGGTGATGCGGTTTTGGCAGTA
CACCAATGGGCGTGGATAGCGGTTTGACTCACGGGGATTTCCAAGTCTCC
ACCCCATTGACGTCAATGGGAGTTTGTTTTGGCACCAAAATCAACGGGAC
TTTCCAAAATGTCGTAATAACCCCGCCCCGTTGACGCAAATGGGCGGTAG
GCGTGTACGGTGGGAGGTCTATATAAGCAGAGGTCGTTTAGTGAACCGTC
AGATCACTAGTAGCTTTATTGCGGTAGTTTATCACAGTTAAATTGCTAAC
GCAGTCAGTGCTCGACTGATCACAGGTAAGTATCAAGGTTACAAGACAGG
TTTAAGGAGGCCAATAGAAACTGGGCTTGTCGAGACAGAGAAGATTCTTG
CGTTTCTGATAGGCACCTATTGGTCTTACTGACATCCACTTTGCCTTTCT
CTCCACAG.

In aspects, the pAAV-SMVP-Flag-CFTR-N-intN may comprise one or more further linker sequences, for example, a third and/or fourth linker sequence. In this aspect, the one or more linker sequences may comprise gggtaccgaagccgctagcgctaccggt (linker, SEQ ID NO: 18) and/or AGAATTAACC (linker, SEQ ID NO: 19).

In aspects, the pAAV-SMVP-Flag-CFTR-N-intN may comprise a tag. For example, the one or more linkers of the preceding paragraph may be operatively bound to a tag such as a Flag tag. The Flag tag may have a sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 10, wherein SEQ ID NO: 10 is

(SEQ ID NO: 10)
ATGGACTACAAAGACCATGACGGTGATTATAAAGATCATGACATCGATTA
CAAGGATGACGATGACAAGCTT.

In aspects, the Flag sequence comprises a nucleic acid sequence that encodes for an amino acid sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 11, wherein SEQ ID NO: 11 is

(SEQ ID NO: 11)
MDYKDHDGDYKDHDIDYKDDDDKL.

The flag sequence (shown in bold italic font) may be operatively connected to the N terminal CFTR fragment (CFTR-N). In aspects, the CFTR-N sequence has at least 90%, or at least 91%, or at least 92%, or at least 93%, or at least 94%, or at least 95%, or at least 96%, or at least 97%, or at least 98%, or at least 99%, or 100% sequence identity to SEQ ID NO: 5, wherein SEQ ID NO: 5 is

(SEQ ID NO: 5)
ATGCAGAGGTCGCCTCTGGAAAAGGCCAGCGTTGTCTCCAAACTTTTTTTCAGCTGGACCA
GACCAATTTTGAGGAAAGGATACAGACAGCGCCTGGAATTGTCAGACATATACCAAATCCC
TTCTGTTGATTCTGCTGACAATCTATCTGAAAAATTGGAAAGAGAATGGGATAGAGAGCTG
GCTTCAAAGAAAAATCCTAAACTCATTAATGCCCTTCGGCGATGTTTTTTCTGGAGATTTAT
GTTCTATGGAATCTTTTTATATTTAGGGGAAGTCACCAAAGCAGTACAGCCTCTCTTA
CTGGGAAGAATCATAGCTTCCTATGACCCGGATAACAAGGAGGAACGCTCTATCGC
GATTTATCTAGGCATAGGCTTATGCCTTCTCTTTATTGTGAGGACACTGCTCCTACAC
CCAGCCATTTTTGGCCTTCATCACATTGGAATGCAGATGAGAATAGCTATGTTTAGT
TTGATTTATAAGAAGACTTTAAAGCTGTCAAGCCGTGTTCTAGATAAAATAAGTATT
GGACAACTTGTTAGTCTCCTTTCCAACAACCTGAACAAATTTGATGAAGGACTTGCA
TTGGCACATTTCGTGTGGATCGCTCCTTTGCAAGTGGCACTCCTCATGGGGCTAATCT
GGGAGTTGTTACAGGCGTCTGCCTTCTGTGGACTTGGTTTCCTGATAGTCCTTGCCCT
TTTTCAGGCTGGGCTAGGGAGAATGATGATGAAGTACAGAGATCAGAGAGCTGGGA
AGATCAGTGAAAGACTTGTGATTACCTCAGAAATGATTGAAAATATCCAATCTGTTA
AGGCATACTGCTGGGAAGAAGCAATGGAAAAAATGATTGAAAACTTAAGACAAAC
AGAACTGAAACTGACTCGGAAGGCAGCCTATGTGAGATACTTCAATAGCTCAGCCTT
CTTCTTCTCAGGGTTCTTTGTGGTGTTTTTATCTGTGCTTCCCTATGCACTAATCAAAG
GAATCATCCTCCGGAAAATATTCACCACCATCTCATTCTGCATTGTTCTGCGCATGGC
GGTCACTCGGCAATTTCCCTGGGCTGTACAAACATGGTATGACTCTCTTGGAGCAAT
AAACAAAATACAGGATTTCTTACAAAAGCAAGAATATAAGACATTGGAATATAACT
TAACGACTACAGAAGTAGTGATGGAGAATGTAACAGCCTTCTGGGAGGAGGGATTT
GGGGAATTATTTGAGAAAGCAAAACAAAACAATAACAATAGAAAAACTTCTAATGG
TGATGACAGCCTCTTCTTCAGTAATTTCTCACTTCTTGGTACTCCTGTCCTGAAAGAT
ATTAATTTCAAGATAGAAAGAGGACAGTTGTTGGCGGTTGCTGGATCCACTGGAGC
AGGCAAGACTTCACTTCTAATGGTGATTATGGGAGAACTGGAGCCTTCAGAGGGTA
AAATTAAGCACAGTGGAAGAATTTCATTCTGTTCTCAGTTTTCCTGGATTATGCCTGG
CACCATTAAAGAAAATATCATCTTTGGTGTTTCCTATGATGAATATAGATACAGAAG
CGTCATCAAAGCATGCCAACTAGAAGAGGACATCTCCAAGTTTGCAGAGAAAGACA
ATATAGTTCTTGGAGAAGGTGGAATCACACTGAGTGGAGGTCAACGAGCAAGAATT
TCTTTAGCAAGAGCAGTATACAAAGATGCTGATTTGTATTTATTAGACTCTCCTTTTG
GATACCTAGATGTTTTAACAGAAAAAGAAATATTTGAAAGCTGTGTCTGTAAACTGA
TGGCTAACAAAACTAGGATTTTGGTCACTTCTAAAATGGAACATTTAAAGAAAGCTG
ACAAAATATTAATTTTGCATGAAGGTAGCAGCTATTTTTATGGGACATTTTCAGAAC
TCCAAAATCTACAGCCAGACTTTAGCTCAAAACTCATGGGATGTGATTCTTTCGACC
AATTTAGTGCAGAAAGAAGAAATTCAATCCTAACTGAGACCTTACACCGTTTCTCAT
TAGAAGGAGATGCTCCTGTCTCCTGGACAGAAACAAAAAAACAATCTTTTAAACAG
ACTGGAGAGTTTGGGGAAAAAAGGAAGAATTCTATTCTCAATCCAATCAACTCTATA
CGAAAATTTTCCATTGTGCAAAAGACTCCCTTACAAATGAATGGCATCGAAGAGGAT
TCTGATGAGCCTTTAGAGAGAAGGCTGTCCTTAGTACCAGATTCTGAGCAGGGAGA
GGCGATACTGCCTCGCATCAGCGTGATCAGCACTGGCCCCACGCTTCAGGCACGAA
GGAGGCAGTCTGTCCTGAACCTGATGACACACTCAGTTAACCAAGGTCAGAACATTC
ACCGAAAGACAACAGCATCCACACGAAAAGTGTCACTGGCCCCTCAGGCAAACTTG
ACTGAACTGGATATATATTCAAGAAGGTTATCTCAAGAAACTGGCTTGGAAATAAGT
GAAGAAATTAACGAAGAAGACTTAAAGGAGTGCTTTTTTGATGATATGGAG.

In aspects, the CFTR-N sequence comprises a nucleic acid sequence that encodes for an amino acid sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 6, wherein SEQ ID NO: 6 is

(SEQ ID NO: 6)
MQRSPLEKASVVSKLFFSWTRPILRKGYRQRLELSDIYQIPSVDSADNLS
EKLEREWDRELASKKNPKLINALRRCFFWRFMFYGIFLYLGEVTKAVQPL
LLGRIIASYDPDNKEERSIAIYLGIGLCLLFIVRTLLLHPAIFGLHHIGM
QMRIAMFSLIYKKTLKLSSRVLDKISIGQLVSLLSNNLNKFDEGLALAHF
VWIAPLQVALLMGLIWELLQASAFCGLGFLIVLALFQAGLGRMMMKYRDQ
RAGKISERLVITSEMIENIQSVKAYCWEEAMEKMIENLRQTELKLTRKAA
YVRYFNSSAFFFSGFFVVFLSVLPYALIKGIILRKIFTTISFCIVLRMAV
TRQFPWAVQTWYDSLGAINKIQDFLQKQEYKTLEYNLTTTEVVMENVTAF
WEEGFGELFEKAKQNNNNRKTSNGDDSLFFSNFSLLGTPVLKDINFKIER
GQLLAVAGSTGAGKTSLLMVIMGELEPSEGKIKHSGRISFCSQFSWIMPG
TIKENIIFGVSYDEYRYRSVIKACQLEEDISKFAEKDNIVLGEGGITLSG
GQRARISLARAVYKDADLYLLDSPFGYLDVLTEKEIFESCVCKLMANKTR
ILVTSKMEHLKKADKILILHEGSSYFYGTFSELQNLQPDFSSKLMGCDSF
DQFSAERRNSILTETLHRFSLEGDAPVSWTETKKQSFKQTGEFGEKRKNS
ILNPINSIRKFSIVQKTPLQMNGIEEDSDEPLERRLSLVPDSEQGEAILP
RISVISTGPTLQARRRQSVLNLMTHSVNQGQNIHRKTTASTRKVSLAPQA
NLTELDIYSRRLSQETGLEISEEINEEDLKECFFDDME.

In aspects, the human CFTR-N region above may be operatively connected to Rhodothermus marinus Intein-N. Intein-N has a sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 1, wherein SEQ ID NO: 1 is

(SEQ ID NO: 1)
TGTCTGGCTGGCGATACTCTCATTACCCTGGCCGATGGACGACGAGTGCC
TATTAGAGAACTGGTGTCACAGCAGAATTTTTCCGTGTGGGCTCTGAATC
CTCAGACTTACCGCCTGGAGAGGGCTAGAGTGAGTAGAGCTTTCTGTACC
GGCATCAAACCTGTGTACCGCCTCACCACTAGACTGGGGAGATCCATTAG
GGCCACTGCCAACCACCGATTTCTCACACCTCAGGGCTGGAAACGAGTCG
ATGAACTCCAGCCTGGAGATTACCTGGCTCTGCCTAGGAGAATCCCTACT
GCCTCCTGA.

In aspects, the Intein N (IntN) sequence comprises a nucleic acid sequence that encodes for an amino acid sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 2, wherein SEQ ID NO: 2 is

(IntN, SEQ ID NO: 2)
CLAGDTLITLADGRRVPIRELVSQQNFSVWALNPQTYRLERARVSRAFCT
GIKPVYRLTTRLGRSIRATANHRFLTPQGWKRVDELQPGDYLALPRRIPT
AS.

In aspects, the pAAV-SMVP-Flag-CFTR-N-intN may comprise a further linker which operatively connects the intein-N sequence and a poly-A signal. For example, the linker may comprise CTCGAGCTCGATGAGTTTGGACAAACCACAACTAGAAT (linker, SEQ ID NO: 20). The SV50 Poly(A) signal may be, for example, a sequence having at least 90%, or at least 91%, or at least 92%, or at least 93%, or at least 94%, or at least 95%, or at least 96%, or at least 97%, or at least 98%, or at least 99%, or 100% sequence identity to that of SEQ ID NO: 21, wherein SEQ ID NO: 21 is

(SEQ ID NO: 21)
GCAGTGAAAAAAATGCTTTATTTGTGAAATTTGTGATGCTATTGCTTTA
TTTGTAACCATTATAAGCTGCAATAAACAAGTT,

which may be further operatively connected to a linker sequence having a sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 22, wherein SEQ ID NO: 22 is:

(SEQ ID NO: 22)
AACAACAACAATTGCATTCATTTTATGTTTCAGGTTCAGGGGGAGGTGT
GGGAGGTTTTTTAAAGCAAGTAAAACCTCTACAAATGTGGTAGCGGCCG
CGGAATGTGTGTCAGTAAAACCTCTACAAATGTGGTAGCGGCCGCAGGT
AGATAAGTAGCATGGCGGGTTAATCATTAACTACA.

In aspects, the pAAV-SMVP-Flag-CFTR-N-intN may comprise an AAV2 ITR sequence. The AAV2 ITR sequence may comprise a sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 23, wherein SEQ ID NO: 23 is

(SEQ ID NO: 23)
AGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCC
CGGGCGGCCTCAGTGAGCGAGCGAGCGCGCAG.

An exemplary ORI-Linker-Promoter-AAV2-Linker-ORI sequence, which may be used in conjunction with a CFTR fragment-Intein sequence is as follows:

(ORI-Linker-Promoter-AAV2-Linker-ORI sequence,
SEQ ID NO: 24)
CCTTAATTAACCTAATTCACTGGCCGTCGTTTTACAACGTCGTGACTGGG
AAAACCCTGGCGTTACCCAACTTAATCGCCTTGCAGCACATCCCCCTTTC
GCCAGCTGGCGTAATAGCGAAGAGGCCCGCACCGATCGCCCTTCCCAACA
GTTGCGCAGCCTGAATGGCGAATGGGACGCGCCCTGTAGCGGCGCATTAA
GCGCGGCGGGTGTGGTGGTTACGCGCAGCGTGACCGCTACACTTGCCAGC
GCCCTAGCGCCCGCTCCTTTCGCTTTCTTCCCTTCCTTTCTCGCCACGTT
CGCCGGCTTTCCCCGTCAAGCTCTAAATCGGGGGCTCCCTTTAGGGTTCC
GATTTAGTGCTTTACGGCACCTCGACCCCAAAAAACTTGATTAGGGTGAT
GGTTCACGTAGTGGGCCATCGCCCTGATAGACGGTTTTTCGCCCTTTGAC
GTTGGAGTCCACGTTCTTTAATAGTGGACTCTTGTTCCAAACTGGAACAA
CACTCAACCCTATCTCGGTCTATTCTTTTGATTTATAAGGGATTTTGCCG
ATTTCGGCCTATTGGTTAAAAAATGAGCTGATTTAACAAAAATTTAACGC
GAATTTTAACAAAATATTAACGTTTATAATTTCAGGTGGCATCTTTCGGG
GAAATGTGCGCGGAACCCCTATTTGTTTATTTTTCTAAATACATTCAAAT
ATGTATCCGCTCATGAGACAATAACCCTGATAAATGCTTCAATAATATTG
AAAAAGGAAGAGTATGAGTATTCAACATTTCCGTGTCGCCCTTATTCCCT
TTTTTGCGGCATTTTGCCTTCCTGTTTTTGCTCACCCAGAAACGCTGGTG
AAAGTAAAAGATGCTGAAGATCAGTTGGGTGCACGAGTGGGTTACATCGA
ACTGGATCTCAATAGTGGTAAGATCCTTGAGAGTTTTCGCCCCGAAGAAC
GTTTTCCAATGATGAGCACTTTTAAAGTTCTGCTATGTGGCGCGGTATTA
TCCCGTATTGACGCCGGGCAAGAGCAACTCGGTCGCCGCATACACTATTC
TCAGAATGACTTGGTTGAGTACTCACCAGTCACAGAAAAGCATCTTACGG
ATGGCATGACAGTAAGAGAATTATGCAGTGCTGCCATAACCATGAGTGAT
AACACTGCGGCCAACTTACTTCTGACAACGATCGGAGGACCGAAGGAGCT
AACCGCTTTTTTGCACAACATGGGGGATCATGTAACTCGCCTTGATCGTT
GGGAACCGGAGCTGAATGAAGCCATACCAAACGACGAGCGTGACACCACG
ATGCCTGTAGTAATGGTAACAACGTTGCGCAAACTATTAACTGGCGAACT
ACTTACTCTAGCTTCCCGGCAACAATTAATAGACTGGATGGAGGCGGATA
AAGTTGCAGGACCACTTCTGCGCTCGGCCCTTCCGGCTGGCTGGTTTATT
GCTGATAAATCTGGAGCCGGTGAGCGTGGGTCTCGCGGTATCATTGCAGC
ACTGGGGCCAGATGGTAAGCCCTCCCGTATCGTAGTTATCTACACGACGG
GGAGTCAGGCAACTATGGATGAACGAAATAGACAGATCGCTGAGATAGGT
GCCTCACTGATTAAGCATTGGTAACTGTCAGACCAAGTTTACTCATATAT
ACTTTAGATTGATTTAAAACTTCATTTTTAATTTAAAAGGATCTAGGTGA
AGATCCTTTTTGATAATCTCATGACCAAAATCCCTTAACGTGAGTTTTCG
TTCCACTGAGCGTCAGACCCCGTAGAAAAGATCAAAGGATCTTCTTGAGA
TCCTTTTTTTCTGCGCGTAATCTGCTGCTTGCAAACAAAAAAACCACCGC
TACCAGCGGTGGTTTGTTTGCCGGATCAAGAGCTACCAACTCTTTTTCCG
AAGGTAACTGGCTTCAGCAGAGCGCAGATACCAAATACTGTCCTTCTAGT
GTAGCCGTAGTTAGGCCACCACTTCAAGAACTCTGTAGCACCGCCTACAT
ACCTCGCTCTGCTAATCCTGTTACCAGTGGCTGCTGCCAGTGGCGATAAG
TCGTGTCTTACCGGGTTGGACTCAAGACGATAGTTACCGGATAAGGCGCA
GCGGTCGGGCTGAACGGGGGGTTCGTGCACACAGCCCAGCTTGGAGCGAA
CGACCTACACCGAACTGAGATACCTACAGCGTGAGCTATGAGAAAGCGC
C.

intC-CFTR-C-HA Construct

In aspects of the dual nucleic acid delivery system, a second construct comprising a second CFTR sequence and a second intein is disclosed. An exemplary second construct is the “intC-CFTR-C-HA” construct. It should be noted, however, that the “HA” portion is optional and may be omitted for clinical administration to an individual. Further disclosed is an intC-CFTR-C-HA construct which may be used for carrying out the disclosed methods. The intC-CFTR-C-HA construct for use in the disclosed compositions and methods may be made by employing routine molecular biology techniques as understood in the art. An exemplary AAV vector is provided at www.addgene.org/browse/sequence/152875/. An exemplary method for preparing an AAV-intein platform containing a gene is described at www.ncbi.nlm.nih.gov/pmc/articles/PMC5374744/. The component sequences for manufacture of the intC-CFTR-C-HA construct and delivery vehicle are described below.

In general, the disclosed constructs employ a human CFTR fragment operatively connected to an Rhodothermus marinus intein. For example, in aspects, the intC-CFTR construct may comprise an intein C operatively connected to a CFTR-C fragment. In aspects, the CFTR-C sequence comprises a nucleic acid sequence having at least 90%, or at least 91%, or at least 92%, or at least 93%, or at least 94%, or at least 95%, or at least 96%, or at least 97%, or at least 98%, or at least 99%, or 100% sequence identity to

(CFTR-C, SEQ ID NO: 7)
AGCATACCAGCAGTGACTACATGGAACACATACCTTCGATATATTACTGT
CCACAAGAGCTTAATTTTTGTGCTAATTTGGTGCTTAGTAATTTTTCTGG
CAGAGGTGGCTGCTTCTTTGGTTGTGCTGTGGCTCCTTGGAAACACTCCT
CTTCAAGACAAAGGGAATAGTACTCATAGTAGAAATAACAGCTATGCAGT
GATTATCACCAGCACCAGTTCGTATTATGTGTTTTACATTTACGTGGGAG
TAGCCGACACTTTGCTTGCTATGGGATTCTTCAGAGGTCTACCACTGGTG
CATACTCTAATCACAGTGTCGAAAATTTTACACCACAAAATGTTACATTC
TGTTCTTCAAGCACCTATGTCAACCCTCAACACGTTGAAAGCAGGTGGGA
TTCTTAATAGATTCTCCAAAGATATAGCAATTTTGGATGACCTTCTGCCT
CTTACCATATTTGACTTCATCCAGTTGTTATTAATTGTGATTGGAGCTAT
AGCAGTTGTCGCAGTTTTACAACCCTACATCTTTGTTGCAACAGTGCCAG
TGATAGTGGCTTTTATTATGTTGAGAGCATATTTCCTCCAAACCTCACAG
CAACTCAAACAACTGGAATCTGAAGGCAGGAGTCCAATTTTCACTCATCT
TGTTACAAGCTTAAAAGGACTATGGACACTTCGTGCCTTCGGACGGCAGC
CTTACTTTGAAACTCTGTTCCACAAAGCTCTGAATTTACATACTGCCAAC
TGGTTCTTGTACCTGTCAACACTGCGCTGGTTCCAAATGAGAATAGAAAT
GATTTTTGTCATCTTCTTCATTGCTGTTACCTTCATTTCCATTTTAACAA
CAGGAGAAGGAGAAGGAAGAGTTGGTATTATCCTGACTTTAGCCATGAAT
ATCATGAGTACATTGCAGTGGGCTGTAAACTCCAGCATAGATGTGGATAG
CTTGATGCGATCTGTGAGCCGAGTCTTTAAGTTCATTGACATGCCAACAG
AAGGTAAACCTACCAAGTCAACCAAACCATACAAGAATGGCCAACTCTCG
AAAGTTATGATTATTGAGAATTCACACGTGAAGAAAGATGACATCTGGCC
CTCAGGGGGCCAAATGACTGTCAAAGATCTCACAGCAAAATACACAGAAG
GTGGAAATGCCATATTAGAGAACATTTCCTTCTCAATAAGTCCTGGCCAG
AGGGTGGGCCTCTTGGGAAGAACTGGATCAGGGAAGAGTACTTTGTTATC
AGCTTTTTTGAGACTACTGAACACTGAAGGAGAAATCCAGATCGATGGTG
TGTCTTGGGATTCAATAACTTTGCAACAGTGGAGGAAAGCCTTTGGAGTG
ATACCACAGAAAGTATTTATTTTTTCTGGAACATTTAGAAAAAACTTGGA
TCCCTATGAACAGTGGAGTGATCAAGAAATATGGAAAGTTGCAGATGAGG
TTGGGCTCAGATCTGTGATAGAACAGTTTCCTGGGAAGCTTGACTTTGTC
CTTGTGGATGGGGGCTGTGTCCTAAGCCATGGCCACAAGCAGTTGATGTG
CTTGGCTAGATCTGTTCTCAGTAAGGCGAAGATCTTGCTGCTTGATGAAC
CCAGTGCTCATTTGGATCCAGTAACATACCAAATAATTAGAAGAACTCTA
AAACAAGCATTTGCTGATTGCACAGTAATTCTCTGTGAACACAGGATAGA
AGCAATGCTGGAATGCCAACAATTTTTGGTCATAGAAGAGAACAAAGTGC
GGCAGTACGATTCCATCCAGAAACTGCTGAACGAGAGGAGCCTCTTCCGG
CAAGCCATCAGCCCCTCCGACAGGGTGAAGCTCTTTCCCCACCGGAACTC
AAGCAAGTGCAAGTCTAAGCCCCAGATTGCTGCTCTGAAAGAGGAGACAG
AAGAAGAGGTGCAAGATACAAGGCTT.

In aspects, the intein-C (intC) sequence is a sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 3, wherein SEQ ID NO: 3 is

(SEQ ID NO: 3)
ATGGCGGCGGCGTGCCCGGAACTGCGTCAGCTGGCGCAGAGCGATGTGT
ATTGGGATCCGATTGTGAGCATTGAACCGGATGGCGTGGAAGAAGTGTT
TGATCTGACCGTGCCGGGCCCGCATAACTTTGTGGCGAACGATATTATT
GCGCATAACTCT.

In aspects, the CFTR-C sequence comprises a nucleic acid sequence that encodes for an amino acid sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 8, wherein SEQ ID NO: 8 is

(SEQ ID NO: 8)
SIPAVTTWNTYLRYITVHKSLIFVLIWCLVIFLAEVAASLVVLWLLGNTP
LQDKGNSTHSRNNSYAVIITSTSSYYVFYIYVGVADTLLAMGFFRGLPLV
HTLITVSKILHHKMLHSVLQAPMSTLNTLKAGGILNRFSKDIAILDDLLP
LTIFDFIQLLLIVIGAIAVVAVLQPYIFVATVPVIVAFIMLRAYFLQTSQ
QLKQLESEGRSPIFTHLVTSLKGLWTLRAFGRQPYFETLFHKALNLHTAN
WFLYLSTLRWFQMRIEMIFVIFFIAVTFISILTTGEGEGRVGIILTLAMN
IMSTLQWAVNSSIDVDSLMRSVSRVFKFIDMPTEGKPTKSTKPYKNGQLS
KVMIIENSHVKKDDIWPSGGQMTVKDLTAKYTEGGNAILENISFSISPGQ
RVGLLGRTGSGKSTLLSAFLRLLNTEGEIQIDGVSWDSITLQQWRKAFGV
IPQKVFIFSGTFRKNLDPYEQWSDQEIWKVADEVGLRSVIEQFPGKLDFV
LVDGGCVLSHGHKQLMCLARSVLSKAKILLLDEPSAHLDPVTYQIIRRTL
KQAFADCTVILCEHRIEAMLECQQFLVIEENKVRQYDSIQKLLNERSLFR
QAISPSDRVKLFPHRNSSKCKSKPQIAALKEETEEEVQDTRL.

The intC-CFTR-C-HA construct may comprise a tag. In aspects, the tag is an HA sequence. The HA sequence can serve as a marker for the detection of the CFTR protein following its delivery to a cell or an individual. In aspects, the HA component is included for experimental applications. In alternative aspects, the HA component is incorporated for therapeutic applications. In aspects where the application is clinical, the construct may be designed without the inclusion of a flag. In scenarios where a HA is used, one example of a suitable HA sequence is TACCCATACGATGTTCCAGATTACGCTTAG (HA SEQ ID NO: 25).

In aspects, the HA sequence comprises a nucleic acid sequence that encodes for an amino acid sequence having at least 90%, or at least 91%, or at least 92%, or at least 93%, or at least 94%, or at least 95%, or at least 96%, or at least 97%, or at least 98%, or at least 99%, or 100% sequence identity to YPYDVPDYA (SEQ ID NO: 26).

In aspects, the intC-CFTR-HA component of the construct may comprise a sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 27, wherein bold italic font indicates the Int-C portion of the construct, and bold indicates the optional “HA” portion of the construct as indicated below:

(SEQ ID NO: 27)
ATG GCG GCG GCG TGC CCG GAA CTG CGT CAG CTG GCG
CAG AGC GAT GTG TAT TGG GAT CCG ATT GTG AGC ATT
GAA CCG GAT GGC GTG GAA GAA GTG TTT GAT CTG ACC
GTG CCG GGC CCG CAT AAC TTT GTG GCG AAC GAT ATT
ATT GCG CAT AAC TCT AGC ATA CCA GCA GTG ACT ACA
TGG AAC ACA TAC CTT CGA TAT ATT ACT GTC CAC AAG
AGC TTA ATT TTT GTG CTA ATT TGG TGC TTA GTA ATT
TTT CTG GCA GAG GTG GCT GCT TCT TTG GTT GTG CTG
TGG CTC CTT GGA AAC ACT CCT CTT CAA GAC AAA GGG
AAT AGT ACT CAT AGT AGA AAT AAC AGC TAT GCA GTG
ATT ATC ACC AGC ACC AGT TCG TAT TAT GTG TTT TAC
ATT TAC GTG GGA GTA GCC GAC ACT TTG CTT GCT ATG
GGA TTC TTC AGA GGT CTA CCA CTG GTG CAT ACT CTA
ATC ACA GTG TCG AAA ATT TTA CAC CAC AAA ATG TTA
CAT TCT GTT CTT CAA GCA CCT ATG TCA ACC CTC AAC
ACG TTG AAA GCA GGT GGG ATT CTT AAT AGA TTC TCC
AAA GAT ATA GCA ATT TTG GAT GAC CTT CTG CCT CTT
ACC ATA TTT GAC TTC ATC CAG TTG TTA TTA ATT GTG
ATT GGA GCT ATA GCA GTT GTC GCA GTT TTA CAA CCC
TAC ATC TTT GTT GCA ACA GTG CCA GTG ATA GTG GCT
TTT ATT ATG TTG AGA GCA TAT TTC CTC CAA ACC TCA
CAG CAA CTC AAA CAA CTG GAA TCT GAA GGC AGG AGT
CCA ATT TTC ACT CAT CTT GTT ACA AGC TTA AAA GGA
CTA TGG ACA CTT CGT GCC TTC GGA CGG CAG CCT TAC
TTT GAA ACT CTG TTC CAC AAA GCT CTG AAT TTA CAT
ACT GCC AAC TGG TTC TTG TAC CTG TCA ACA CTG CGC
TGG TTC CAA ATG AGA ATA GAA ATG ATT TTT GTC ATC
TTC TTC ATT GCT GTT ACC TTC ATT TCC ATT TTA ACA
ACA GGA GAA GGA GAA GGA AGA GTT GGT ATT ATC CTG
ACT TTA GCC ATG AAT ATC ATG AGT ACA TTG CAG TGG
GCT GTA AAC TCC AGC ATA GAT GTG GAT AGC TTG ATG
CGA TCT GTG AGC CGA GTC TTT AAG TTC ATT GAC ATG
CCA ACA GAA GGT AAA CCT ACC AAG TCA ACC AAA CCA
TAC AAG AAT GGC CAA CTC TCG AAA GTT ATG ATT ATT
GAG AAT TCA CAC GTG AAG AAA GAT GAC ATC TGG CCC
TCA GGG GGC CAA ATG ACT GTC AAA GAT CTC ACA GCA
AAA TAC ACA GAA GGT GGA AAT GCC ATA TTA GAG AAC
ATT TCC TTC TCA ATA AGT CCT GGC CAG AGG GTG GGC
CTC TTG GGA AGA ACT GGA TCA GGG AAG AGT ACT TTG
TTA TCA GCT TTT TTG AGA CTA CTG AAC ACT GAA GGA
GAA ATC CAG ATC GAT GGT GTG TCT TGG GAT TCA ATA
ACT TTG CAA CAG TGG AGG AAA GCC TTT GGA GTG ATA
CCA CAG AAA GTA TTT ATT TTT TCT GGA ACA TTT AGA
AAA AAC TTG GAT CCC TAT GAA CAG TGG AGT GAT CAA
GAA ATA TGG AAA GTT GCA GAT GAG GTT GGG CTC AGA
TCT GTG ATA GAA CAG TTT CCT GGG AAG CTT GAC TTT
GTC CTT GTG GAT GGG GGC TGT GTC CTA AGC CAT GGC
CAC AAG CAG TTG ATG TGC TTG GCT AGA TCT GTT CTC
AGT AAG GCG AAG ATC TTG CTG CTT GAT GAA CCC AGT
GCT CAT TTG GAT CCA GTA ACA TAC CAA ATA ATT AGA
AGA ACT CTA AAA CAA GCA TTT GCT GAT TGC ACA GTA
ATT CTC TGT GAA CAC AGG ATA GAA GCA ATG CTG GAA
TGC CAA CAA TTT TTG GTC ATA GAA GAG AAC AAA GTG
CGG CAG TAC GAT TCC ATC CAG AAA CTG CTG AAC GAG
AGG AGC CTC TTC CGG CAA GCC ATC AGC CCC TCC GAC
AGG GTG AAG CTC TTT CCC CAC CGG AAC TCA AGC AAG
TGC AAG TCT AAG CCC CAG ATT GCT GCT CTG AAA GAG
GAG ACA GAA GAA GAG GTG CAA GAT ACA AGG CTT TAC
CCA TAC GAT GTT CCA GAT TAC GCT TAG.

In aspects, the intC-CFTR-HA sequences comprises a nucleic acid sequence that encodes for an amino acid sequence having at least 90%, at least 91%, or at least 92%, or at least 93%, or at least 94%, or at least 95%, or at least 96%, or at least 97%, or at least 98%, or at least 99%, or 100% sequence identity to SEQ ID NO: 28, wherein SEQ ID NO: 28 is:

(SEQ ID NO: 28)
M A A A C P E L R Q L A Q S D V Y W D P I V S I E
P D G V E E V F D L T V P G P H N F V A N D I I A
H N S S I P A V T T W N T Y L R Y I T V H K S L I
F V L I W C L V I F L A E V A A S L V V L W L L G
N T P L Q D K G N S T H S R N N S Y A V I I T S T
S S Y Y V F Y I Y V G V A D T L L A M G F F R G L
P L V H T L I T V S K I L H H K M L H S V L Q A P
M S T L N T L K A G G I L N R F S K D I A I L D D
L L P L T I F D F I Q L L L I V I G A I A V V A V
L Q P Y I F V A T V P V I V A F I M L R A Y F L Q
T S Q Q L K Q L E S E G R S P I F T H L V T S L K
G L W T L R A F G R Q P Y F E T L F H K A L N L H
T A N W F L Y L S T L R W F Q M R I E M I F V I F
F I A V T F I S I L T T G E G E G R V G I I L T L
A M N I M S T L Q W A V N S S I D V D S L M R S V
S R V F K F I D M P T E G K P T K S T K P Y K N G
Q L S K V M I I E N S H V K K D D I W P S G G Q M
T V K D L T A K Y T E G G N A I L E N I S F S I S
P G Q R V G L L G R T G S G K S T L L S A F L R L
L N T E G E I Q I D G V S W D S I T L Q Q W R K A
F G V I P Q K V F I F S G T F R K N L D P Y E Q W
S D Q E I W K V A D E V G L R S V I E Q F P G K L
D F V L V D G G C V L S H G H K Q L M C L A R S V
L S K A K I L L L D E P S A H L D P V T Y Q I I R
R T L K Q A F A D C T V I L C E H R I E A M L E C
Q Q F L V I E E N K V R Q Y D S I Q K L L N E R S
L F R Q A I S P S D R V K L F P H R N S S K C K S
K P Q I A A L K E E T E E E V Q D T R L Y P Y D V
P D Y A.

pAAV-SMVP-intC-CFTR-C-HA

In aspects, the pAAV-SMVP-intC-CFTR-C-HA construct may comprise an Origin of Replication (ORI) sequence. The ORI sequence may have at least 90%, or at least 91%, or at least 92%, or at least 93%, or at least 94%, or at least 95%, or at least 96%, or at least 97%, or at least 98%, or at least 99%, or 100% sequence identity to ACGCTTCCCGAAGGGAGAAAGGCGGACAGGTATCCGGTAAGCGGCAGGGTCGGAA CAGGAGAGCGCACGAGGGAGCTTCCAGGGGGAAACGCCTGGTATCTTTATAGTCCT GTCGGGTTTCGCCACCTCTGACTTGAGCGTCGATTTTTGTGATGCTCGTCAGGGGGG CGGAGCCTATGGAA (SEQ ID NO: 14).

In aspects, the pAAV-SMVP-intC-CFTR-C-HA construct may comprise a linker sequence. In aspects, the linker sequence may be AAACGCCAGCAACGCGGCCTTTTTACGGTTCCTGGCCTTTTGCTGCGGTTTTGCTCAC ATGTTCTTTCCTGCGTTATCCCCTGATTCTGTGGATAACCGTATTACCGCCTTTGAGT GAGCTGATACCGCTCGCCGCAGCCGAACGACCGAGCGCAGCGAGTCAGTGAGCGAG GAAGCGGAAGAGCGCCCAATACGCAAACCGCCTCTCCCCGCGCGTTGGCCGATTCA TTAATGCAGCTGGCACGACAGGTTTCCCGACTGGAAAGCGGGCAGTGAGCGCAACG CAATTAATGTGAGTTAGCTCACTCATTAGGCACCCCAGGCTTTACACTTTATGCTTCC GGCTCGTATGTTGTGTGGAATTGTGAGCGGATAACAATTTCACACAGGAAACAGCTA TGACCATGATTACGCCAGATTTAATTAAGG (SEQ ID NO: 15). In this aspect, the linker sequence may be used to link the ORI sequence to the AAV 2 ITR sequence.

In aspects, the pAAV-SMVP-intC-CFTR-C-HA may comprise an AAV 2 ITR sequence. AAV 2 ITR (inverted terminal repeat) sequences may be provided for AAV vector replication and expression of the CFTR gene fragment. In this aspect, the AAV 2ITR sequence may comprise SEQ ID NO:

(5′ AAV 2 ITR, SEQ ID NO: 16)
CTGCGCGCTCGCTCGCTCACTGAGGCCGCCCGGGCAAAGCCCGGGCGTC
GGGCGACCTTTGGTCGCCCGGCCTCAGTGAGCGAGCGAGCGCGCAGAGA
GGGAGTGGCCAACTCCATCACTAGGGGTTCCT.

In aspects, the pAAV-SMVP-intC-CFTR-C-HA may comprise a second linker sequence. The second linker sequence may be used to link the AAV 2 ITR sequence and the SMVP promoter sequence. In this aspect, the second linker sequence may comprise SEQ ID NO: 17, wherein SEQ ID NO: 17 is

(SEQ ID NO: 17)
TGTAGTTAATGATTAACCCGCCATGCTACTTATCTACCTGCGGCCGCGGA
ATGTGTGTCAGTTAGGGTGTGGAAAGTCCCCAGGCTCCCCAGCAGGCAGA
AGTATGCAAAGCATGCATCTCAATTAGTCAGCAACCAGTCCCGGTCTCCT
CCCATGCATGTCAATATTGGCCATTAGCCATATTATTCATTGGTTATATA
GCATAAATCAATATTGGCTATTGGCCATTGCATACGTTGTATCTATATCA
TAATATGTACATTTATATTGGCTCATGTCCAATATGACCGCCATGTT.

In aspects, the pAAV-SMVP-intC-CFTR-C-HA may comprise an SMVP promoter sequence. In this aspect, the SMVP promoter sequence may comprise SEQ ID NO: 9, wherein SEQ ID NO: 9 is

(SEQ ID NO: 9)
GGCATTGATTATTGACTAGTTATTAATAGTAATCAATTACGGGGTCATTA
GTTCATAGCCCATATATGGAGTTCCGCGTTACATAACTTACGGTAAATGG
CCCGCCTGGCTGACCGCCCAACGACCCCCGCCCATTGACGTCAATAATGA
CGTATGTTCCCATAGTAACGCCAATAGGGACTTTCCATTGACGTCAATGG
GTGGAGTATTTACGGTAAACTGCCCACTTGGCAGTACATCAAGTGTATCA
TATGCCAAGTCCGCCCCCTATTGACGTCAATGACGGTAAATGGCCCGCCT
GGCATTATGCCCAGTACATGACCTTACGGGACTTTCCTACTTGGCAGTAC
ATCTACGTATTAGTCATCGCTATTACCATGGTGATGCGGTTTTGGCAGTA
CACCAATGGGCGTGGATAGCGGTTTGACTCACGGGGATTTCCAAGTCTCC
ACCCCATTGACGTCAATGGGAGTTTGTTTTGGCACCAAAATCAACGGGAC
TTTCCAAAATGTCGTAATAACCCCGCCCCGTTGACGCAAATGGGCGGTAG
GCGTGTACGGTGGGAGGTCTATATAAGCAGAGGTCGTTTAGTGAACCGTC
AGATCACTAGTAGCTTTATTGCGGTAGTTTATCACAGTTAAATTGCTAAC
GCAGTCAGTGCTCGACTGATCACAGGTAAGTATCAAGGTTACAAGACAGG
TTTAAGGAGGCCAATAGAAACTGGGCTTGTCGAGACAGAGAAGATTCTTG
CGTTTCTGATAGGCACCTATTGGTCTTACTGACATCCACTTTGCCTTTCT
CTCCACAG.

In aspects, the pAAV-SMVP-intC-CFTR-C-HA may comprise a third linker sequence. The third linker sequence may be used to link the SMVP promoter sequence and a fourth linker. In this aspect, the second third sequence may comprise GGGTACCGAAGCCGCTAGCGCTACCGGT (SEQ ID NO: 18).

In aspects, the pAAV-SMVP-intC-CFTR-C-HA may comprise a fourth linker sequence. The fourth linker sequence may be used to link the SMVP promoter sequence and the intein C sequence. In this aspect, the fourth sequence may comprise CGCCACC.

In aspects, the C-intein is a sequence having at least 90%, or at least 91%, or at least 92%, or at least 93%, or at least 94%, or at least 95%, or at least 96%, or at least 97%, or at least 98%, or at least 99%, or 100% sequence identity to SEQ ID NO: 3, as set forth above.

The intC sequence may be operatively connected to the C terminal CFTR fragment (CFTR-C). In aspects, the CFTR-C sequence has at least 90%, or at least 91%, or at least 92%, or at least 93%, or at least 94%, or at least 95%, or at least 96%, or at least 97%, or at least 98%, or at least 99%, or 100% sequence identity to CFTR-C, SEQ ID NO: 7, as set forth above.

The CFTR-C sequence may be operatively connected to a tag. The tag may be hemagglutinin (“HA”). In aspects, the HA sequence has at least 90%, or at least 91%, or at least 92%, or at least 93%, or at least 94%, or at least 95%, or at least 96%, or at least 97%, or at least 98%, or at least 99%, or 100% sequence identity to TACCCATACGATGTTCCAGATTACGCTTAG (HA, SEQ ID NO: 25), or the sequence encoding for the protein, YPYDVPDYA (HA, SEQ JD NO: 26).

In aspects, the HA tag may be joined to a SV40 polyA signal via a linker. For example, the linker may comprise CTCGAGCTCGATGAGTTTGGACAAACCACAACTAGAAT (linker, SEQ ID NO: 20). The SV40 poly(A) signal may have the sequence gcagtgaaaaaaatgctttatttgtgaaatttgtgatgctattgctttatttgtaaccattataagctgcaataaacaagtt (SV40 poly(A) signal SEQ ID NO: 21). This sequence may be further operatively connected to a further linker having the sequence

(linker, SEQ ID NO: 22)
AACAACAACAATTGCATTCATTTTATGTTTCAGGTTCAGGGGGAGGTGTG
GGAGGTTTTTTAAAGCAAGTAAAACCTCTACAAATGTGGTAGCGGCCGCG
GAATGTGTGTCAGTAAAACCTCTACAAATGTGGTAGCGGCCGCAGGTAGA
TAAGTAGCATGGCGGGTTAATCATTAACTACA,

which may be further operatively connected to the AAV2 ITR, having the sequence

(3′ AAV2 ITR SEQ ID NO: 23)
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAG.

An exemplary ORI-Linker-Promoter-AAV2-Linker-ORI sequence, which may be used in conjunction with a CFTR fragment-Intein sequence is as follows:

(SEQ ID NO: 24)
CCTTAATTAACCTAATTCACTGGCCGTCGTTTTACAACGTCGTGACTGGG
AAAACCCTGGCGTTACCCAACTTAATCGCCTTGCAGCACATCCCCCTTTC
GCCAGCTGGCGTAATAGCGAAGAGGCCCGCACCGATCGCCCTTCCCAACA
GTTGCGCAGCCTGAATGGCGAATGGGACGCGCCCTGTAGCGGCGCATTAA
GCGCGGCGGGTGTGGTGGTTACGCGCAGCGTGACCGCTACACTTGCCAGC
GCCCTAGCGCCCGCTCCTTTCGCTTTCTTCCCTTCCTTTCTCGCCACGTT
CGCCGGCTTTCCCCGTCAAGCTCTAAATCGGGGGCTCCCTTTAGGGTTCC
GATTTAGTGCTTTACGGCACCTCGACCCCAAAAAACTTGATTAGGGTGAT
GGTTCACGTAGTGGGCCATCGCCCTGATAGACGGTTTTTCGCCCTTTGAC
GTTGGAGTCCACGTTCTTTAATAGTGGACTCTTGTTCCAAACTGGAACAA
CACTCAACCCTATCTCGGTCTATTCTTTTGATTTATAAGGGATTTTGCCG
ATTTCGGCCTATTGGTTAAAAAATGAGCTGATTTAACAAAAATTTAACGC
GAATTTTAACAAAATATTAACGTTTATAATTTCAGGTGGCATCTTTCGGG
GAAATGTGCGCGGAACCCCTATTTGTTTATTTTTCTAAATACATTCAAAT
ATGTATCCGCTCATGAGACAATAACCCTGATAAATGCTTCAATAATATTG
AAAAAGGAAGAGTATGAGTATTCAACATTTCCGTGTCGCCCTTATTCCCT
TTTTTGCGGCATTTTGCCTTCCTGTTTTTGCTCACCCAGAAACGCTGGTG
AAAGTAAAAGATGCTGAAGATCAGTTGGGTGCACGAGTGGGTTACATCGA
ACTGGATCTCAATAGTGGTAAGATCCTTGAGAGTTTTCGCCCCGAAGAAC
GTTTTCCAATGATGAGCACTTTTAAAGTTCTGCTATGTGGCGCGGTATTA
TCCCGTATTGACGCCGGGCAAGAGCAACTCGGTCGCCGCATACACTATTC
TCAGAATGACTTGGTTGAGTACTCACCAGTCACAGAAAAGCATCTTACGG
ATGGCATGACAGTAAGAGAATTATGCAGTGCTGCCATAACCATGAGTGAT
AACACTGCGGCCAACTTACTTCTGACAACGATCGGAGGACCGAAGGAGCT
AACCGCTTTTTTGCACAACATGGGGGATCATGTAACTCGCCTTGATCGTT
GGGAACCGGAGCTGAATGAAGCCATACCAAACGACGAGCGTGACACCACG
ATGCCTGTAGTAATGGTAACAACGTTGCGCAAACTATTAACTGGCGAACT
ACTTACTCTAGCTTCCCGGCAACAATTAATAGACTGGATGGAGGCGGATA
AAGTTGCAGGACCACTTCTGCGCTCGGCCCTTCCGGCTGGCTGGTTTATT
GCTGATAAATCTGGAGCCGGTGAGCGTGGGTCTCGCGGTATCATTGCAGC
ACTGGGGCCAGATGGTAAGCCCTCCCGTATCGTAGTTATCTACACGACGG
GGAGTCAGGCAACTATGGATGAACGAAATAGACAGATCGCTGAGATAGGT
GCCTCACTGATTAAGCATTGGTAACTGTCAGACCAAGTTTACTCATATAT
ACTTTAGATTGATTTAAAACTTCATTTTTAATTTAAAAGGATCTAGGTGA
AGATCCTTTTTGATAATCTCATGACCAAAATCCCTTAACGTGAGTTTTCG
TTCCACTGAGCGTCAGACCCCGTAGAAAAGATCAAAGGATCTTCTTGAGA
TCCTTTTTTTCTGCGCGTAATCTGCTGCTTGCAAACAAAAAAACCACCGC
TACCAGCGGTGGTTTGTTTGCCGGATCAACAGCTACCAACTCTTTTTCCG
AAGGTAACTGGCTTCAGCAGAGCGCAGATACCAAATACTGTCCTTCTAGT
GTAGCCGTAGTTAGGCCACCACTTCAAGAACTCTGTAGCACCGCCTACAT
ACCTCGCTCTGCTAATCCTGTTACCAGTGGCTGCTGCCAGTGGCGATAAG
TCGTGTCTTACCGGGTTGGACTCAAGACGATAGTTACCGGATAAGGCGCA
GCGGTCGGGCTGAACGGGGGGTTCGTGCACACAGCCCAGCTTGGAGCGAA
CGACCTACACCGAACTGAGATACCTACAGCGTGAGCTATGAGAAAGCGC
C.

### Pharmaceutical Compositions

The present invention also concerns pharmaceutical compositions comprising the vector system or the viral vector system or the host cells of the invention optionally in combination with a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilizing agent(s), and other carrier agents that may aid or increase the viral entry into the target site (such as for example a lipid delivery system). The construct or vector can be administered in vivo or ex vivo.

Pharmaceutical compositions adapted for topical or parenteral administration, comprising an amount of a compound, constitute a preferred embodiment of the invention. For parenteral administration, the compositions may be best used in the form of a sterile aqueous solution which may contain other substances, for example enough salts or monosaccharides to make the solution isotonic with blood. The pharmaceutical composition of the present invention may be delivered to the retina preferentially via the subretinal injection or it can also be prepared in the form of injectable suspension, eye lotion or ophthalmic ointment that can be delivered to the retina with a non-invasive procedure.

The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

### Gene Therapy Techniques and Methods

The present invention also provides a pharmaceutical composition for treating an individual by gene therapy, wherein the composition comprises a therapeutically effective amount of the vector system or viral vector system or host cell of the present invention comprising one or more deliverable therapeutic and/or diagnostic transgenes(s) or a viral particle produced by or obtained from same. The pharmaceutical composition may be for human or animal usage.

Typically, an ordinary skilled clinician will determine the actual dosage which will be most suitable for an individual subject and it will vary with the age, weight and response of the particular individual and administration route. A dose range between 1×10¹⁰ and 1×10¹⁵ genome copies of each vector/kg, such as between 1×10¹¹ and 1×10¹³ genome copies of each vector/kg are expected to be effective in humans.

Dosage regimes and effective amounts to be administered can be determined by ordinarily skilled clinicians. Administration may be in the form of a single dose or multiple doses. General methods for performing gene therapy using polynucleotides, expression constructs, and vectors are known in the art (see, for example, Gene Therapy: Principles and Applications, Springer Verlag 1999; and U.S. Pat. Nos. 6,461,606; 6,204,251 and 6,106,826). The subject invention also concerns methods for expressing a selected polypeptide in a cell. In one embodiment, the method comprises incorporating in the cell the vector system of the invention that comprises polynucleotide sequences encoding the selected polypeptide and expressing the polynucleotide sequences in the cell. The selected polypeptide can be one that is heterologous to the cell. In one embodiment, the cell is a mammalian cell. In one embodiment, the cell is a human cell.

In one aspect, delivery of the compositions may be via intranasal delivery. In other aspects, the delivery may be via pulmonary delivery.

For example, disclosed are methods for the intranasal administration of the disclosed compositions. In aspects, the delivery of the composition is directly into the nasal cavity of a subject. The active compound may be formulated in a suitable pharmaceutical composition, such as a nasal spray, which can be easily administered using a standard nasal delivery device. The intranasal administration route is particularly advantageous for the delivery of viral vectors, as it bypasses the potential for systemic side effects and allows for targeted delivery to respiratory tissues.

In other aspects, disclosed are methods for the pulmonary administration of the disclosed compositions. The disclosed compositions may be inhaled into the lungs of a subject. This route of administration allows for the direct delivery of the active compound to the respiratory tract, facilitating localized treatment and potentially reducing systemic side effects. The active compound may be formulated in a suitable pharmaceutical composition, such as an aerosol or dry powder, which can be administered using a standard inhaler or nebulizer. The pulmonary administration route is particularly advantageous for the delivery of viral vectors, as it allows for targeted delivery to the lung tissues.

### Kits

The subject invention also concerns kits comprising the construct system or viral vector system or the host cells of the invention in one or more containers. Kits of the invention can optionally include pharmaceutically acceptable carriers and/or diluents. In one embodiment, a kit of the invention includes one or more other components, adjuncts, or adjuvants as described herein. In one embodiment, a kit of the invention includes instructions or packaging materials that describe how to administer a vector system of the kit. Containers of the kit can be of any suitable material, e.g., glass, plastic, metal, etc., and of any suitable size, shape, or configuration. In one embodiment, the construct system or viral vector system or the host cells of the invention is provided in the kit as a solid. In another embodiment, the construct system or viral vector system or the host cells of the invention is provided in the kit as a liquid or solution. In one embodiment, the kit comprises an ampoule or syringe containing the construct system or viral vector system or the host cells of the invention in liquid or solution form.

### Cell Systems

In a further aspect, disclosed is a cell comprising the construct system as disclosed herein. For example, the cell may be a bacterial, yeast, plant, or mammalian cells which comprises at least one of the construct comprising a nucleic acid sequence corresponding to a first portion of a CFTR gene and a nucleic acid sequence corresponding to a an intein gene, as described herein.

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

### Example 1

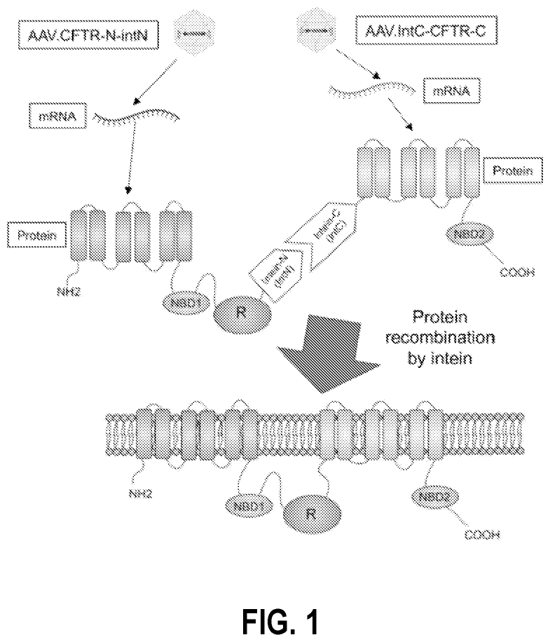

Cystic Fibrosis (CF) is a lethal genetic disease resulting in multiorgan dysfunction that is caused by loss of function of the anion channel CFTR. Delivery of functional CFTR into diseased organs is an ideal approach to treat CF. Success in this approach has been limited by the large size of the CFTR gene, inability to deliver a construct through the thick airway mucus of CF, and lack of durability in construct expression in the airway epithelium. A novel CFTR delivery system was developed using adeno-associated virus (AAV) and intein technology, which allows for the delivery of a split CFTR gene using two distinct AAV constructs. These two transcripts are then translated into split proteins, which combine by inteins into a fully functional, recombined CFTR protein (FIG. 1). The approach has been validated in patient-derived primary human nasal epithelial cells from six people with CF (PwCF) who are insensitive to CFTR modulators, with CFTR rescue confirmed by ion transport studies. This combined AAV (serotype 6) approach targeting progenitor cell populations of airway cells from PwCF who are refractory to CFTR modulators has restored their CFTR activity, and therefore is an AAV serotype that can achieve apical infection of primary airway cells.

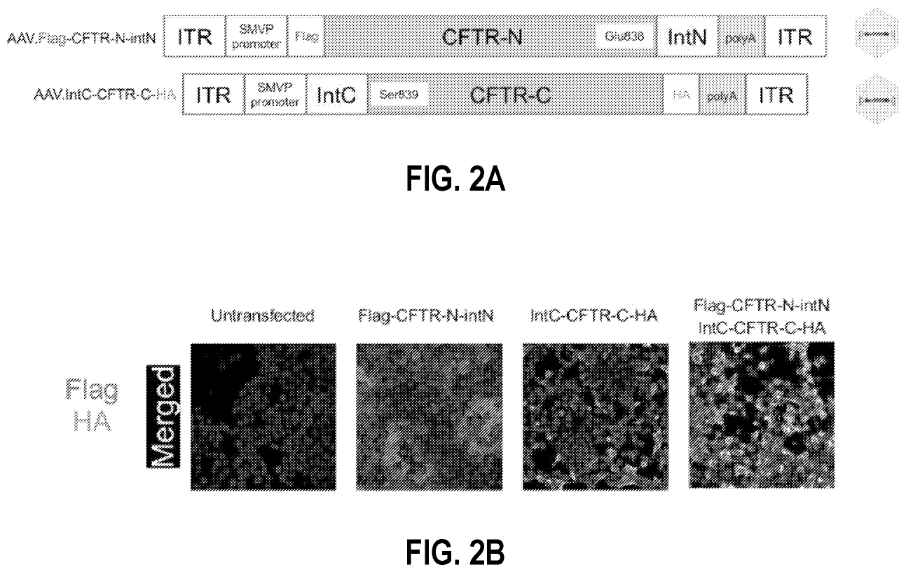

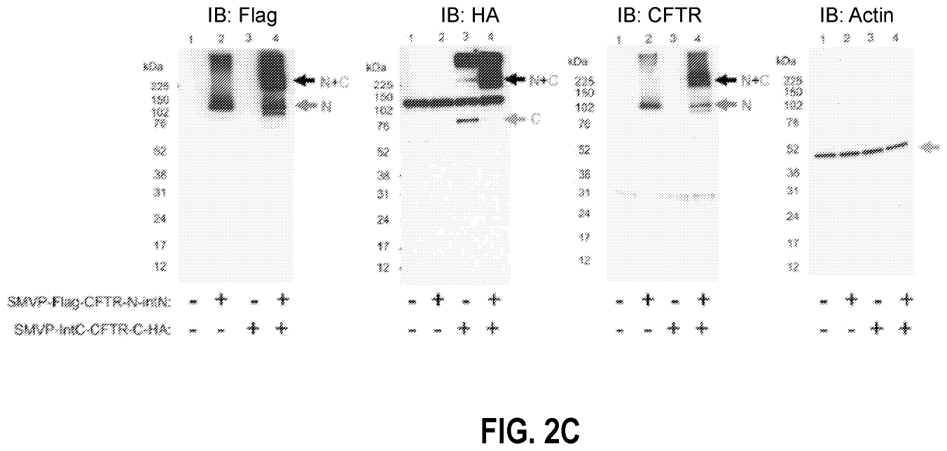

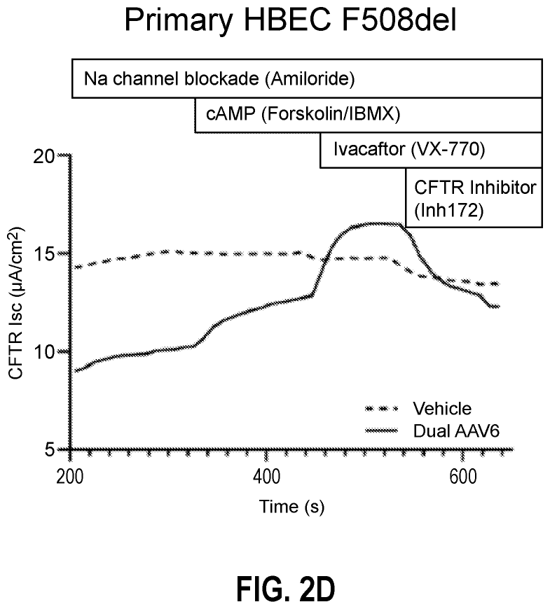

To pursue this approach, functional split CFTR-fused inteins (N and C) are created and packaged into AAVs capable of infecting patient-derived airway epithelial cells, ultimately producing recombined intact CFTR in the cells. To achieve this, the CFTR cDNA was split and an intein coding fragment (IntN or IntC from the eubacterium Rhodothermus marinus) attached to each CFTR fragment. The split fusion genes were then packaged into AAV plasmids which are termed AAV.CFTR-N-intN and AAV.intC-CFTR-C (FIG. 2A; named based on which half of the CFTR cDNA is included). Linker DNA between CFTR and intein (QuikChange II XL Site-Mutagenesis Kit [Agilent]) was removed to minimize DNA length and avoid potential protein dysfunction due to inserted amino acids from the linker. Additionally, AAV.CFTR-N-intN was tagged with “Flag” and AAV.intC-CFTR-C with “HA” to identify the fraction of cells co-expressing both CFTR.N-intN and intC-CFTR-C, as confirmed by immunofluorescence in 293T cells (FIG. 2B). These plasmids were packaged into two AAV6 (serotype 6) vectors. Infection with these AAV6 vectors produced a full-size CFTR protein in AAV6-infected 293T cells (FIG. 2C). Patient-derived F508del homozygous primary HBECs were transfected at the proliferating progenitor state in solution (after trypsin) with these AAV6 vectors and grown at air-liquid interface to be differentiated. Notably, CFTR activity was restored in these F508del homozygous primary HBECs four weeks after AAV6 infection (FIG. 2D), suggesting that this dual AAV approach may be applicable to rescue the function of CFTR in people with CF regardless of CFTR genotype, especially those who are insensitive to CFTR modulators.

AAV6.2FF with split CFTRs (dual AAV) restores CFTR function in vitro in human nasal epithelial (HNE) cells from people with CF (PwCF) who are insensitive to CFTR modulators

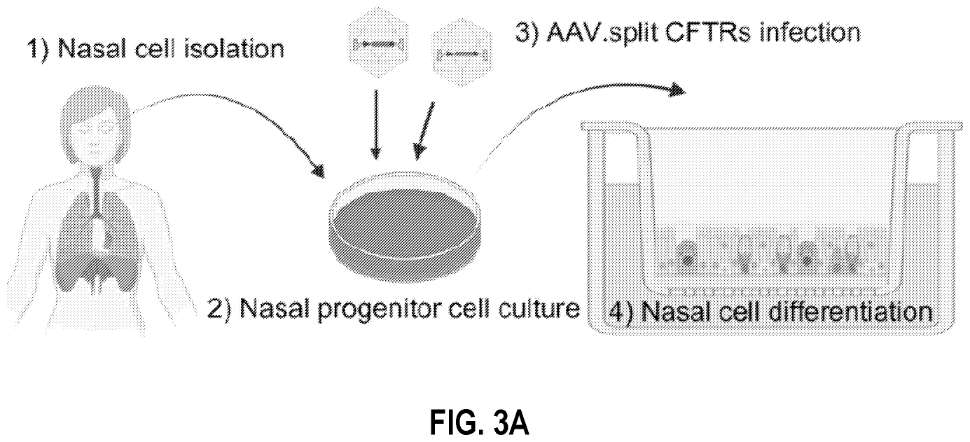

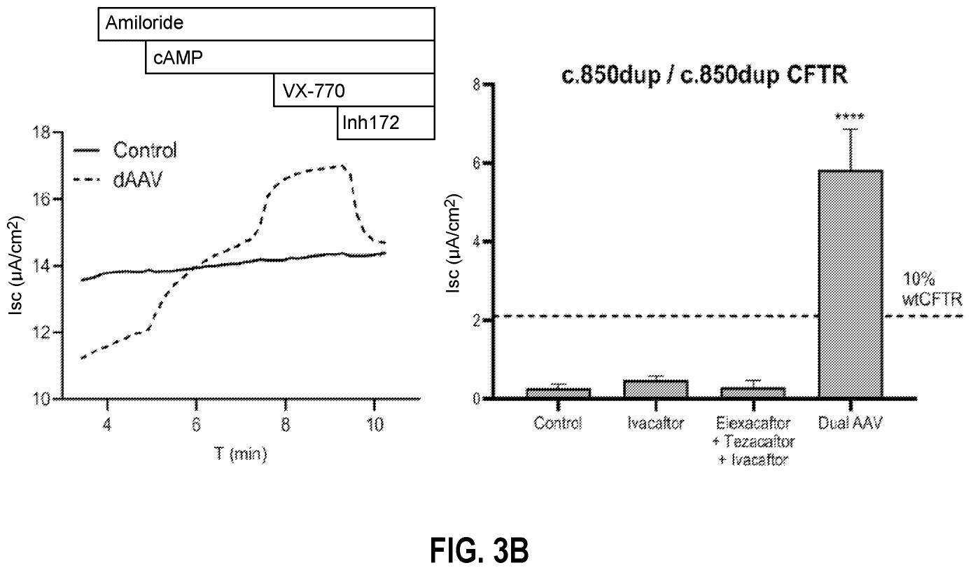

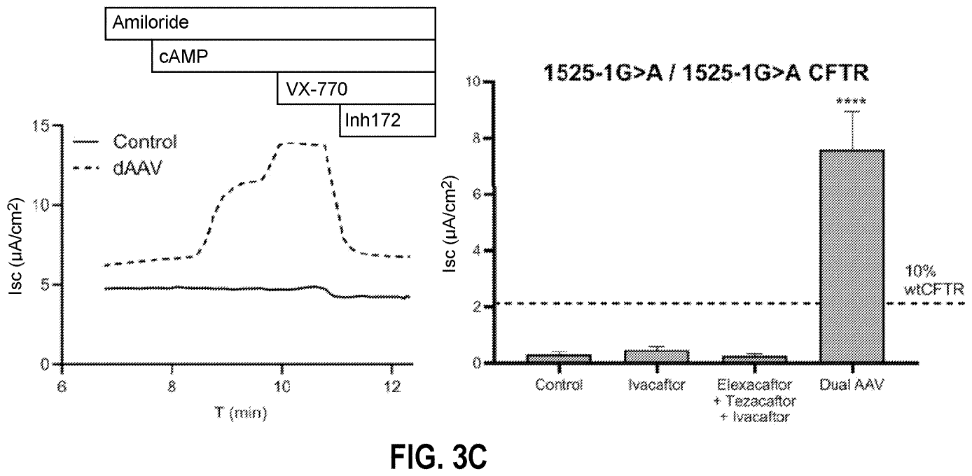

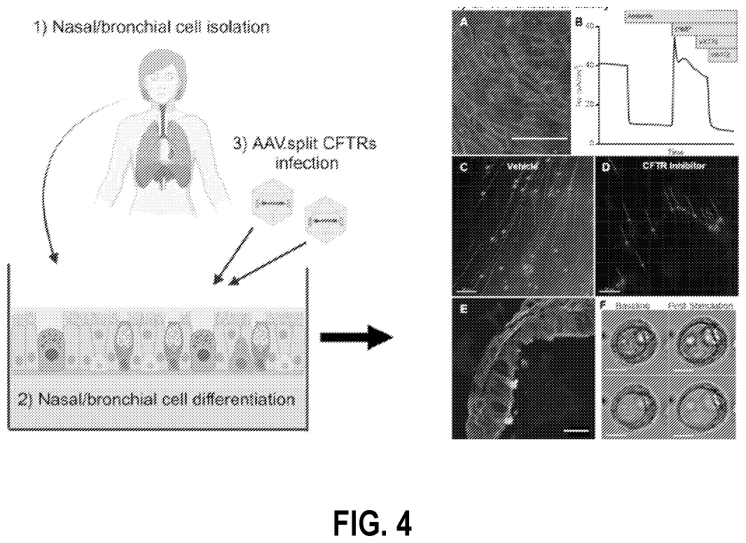

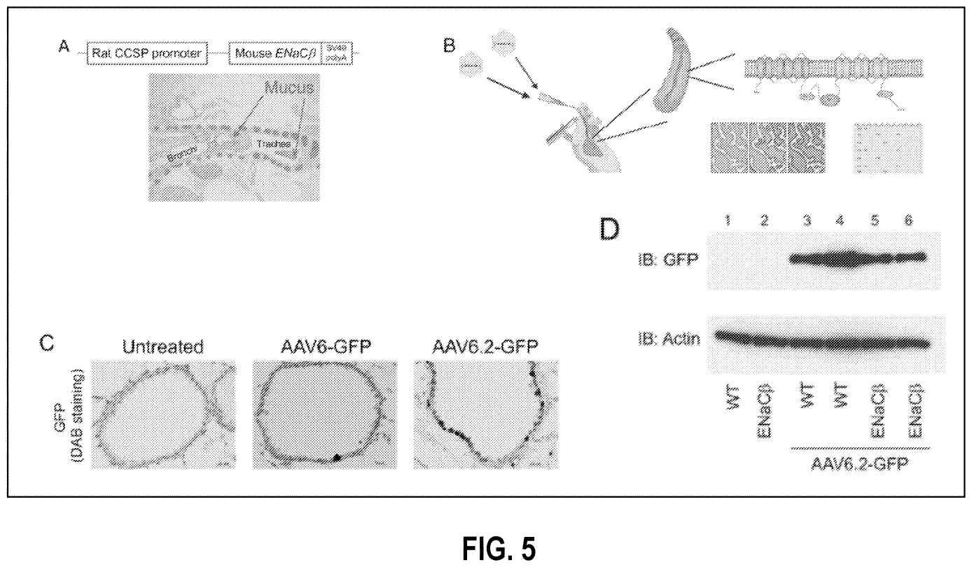

Applicant infected HNE cells, which are in a proliferating progenitor state, from PwCF homozygous for c.850dup or 1525-1G>A CFTR with AAV6.2FF containing split CFTRs (Dual AAV) and grew them at air-liquid interface to a differentiated state (FIG. 3A). AAV6.2FF, which is a new version of AAV6 (F129L, Y445F and Y731F) that can transduce lung epithelial cells, including airway epithelial cells, better than AAV632 was created using QuikChange II XL Site-Mutagenesis Kit (Agilent). In vitro physiological CFTR function was assessed four weeks after infection. Notably, the physiological function of CFTR was restored in the HNE cells homozygous for c.850dup (FIG. 3B) or 1525-1G>A CFTR (FIG. 3C), neither of which are responsive to CFTR modulators including HEMT (elexacaftor/tezacaftor/ivacaftor) (FIG. 3B, 3C). Similar results have been achieved in cells from four additional subjects, all non-responsive to HEMT. These data suggest that the dual AAV approach is effective if it can be delivered to appropriately differentiated airway epithelial cells by apical infection, which mimics a future therapeutic approach using intranasal or intratracheal administration.

### Example 2

Improvement of CFTR function to >10% of wild-type has historically been utilized as a marker of clinical response in cell models; for example, this threshold was utilized by Fred van Goor and colleagues in characterizing the response of rare CFTR variants to ivacaftor in a Fisher Rat Thyroid model (Van Goor F, Yu H, Burton B, Hoffman B J. Effect of ivacaftor on CFTR forms with missense mutations associated with defects in protein processing or function. Journal of cystic fibrosis: official journal of the European Cystic Fibrosis Society 2014; 13: 29-36). This is particularly notable, as the data from that study was used by Vertex Pharmaceuticals to obtain FDA label expansion for ivacaftor to include individuals harboring the CFTR variants. This threshold, therefore, has been acknowledged by both the scientific community and the FDA as representing an in vitro measure that predicts clinical efficacy.

Culture Methods. HNEs were obtained by non-invasive nasal brushing, processed, and expanded in P100 dishes as previously described (Brewington J J, Filbrandt E T, LaRosa F J, 3rd, Moncivaiz J D, Ostmann A J, Strecker L M, Clancy J P. Brushed nasal epithelial cells are a surrogate for bronchial epithelial CFTR studies. JCI insight 2018; 3: e99385.). Once cells reached 80% confluence, they were passaged by adding 0.1% trypsin for 5 minutes to facilitate cell detachment and manual detachment with a cell scraper. The cell mix was centrifuged and the pellet reconstituted in media and counted. HNEs were then seeded onto Transwell®-Clear permeable supports (0.33 cm2 filters, 0.4 μm pore size) pre-coated with type IV collagen at approximately 260,000 cells/cm2 (80,000 cells/insert). AAV vectors were then added directly to the cell compartment of the Transwell. All cells were maintained in Differentiation Media, changing daily, removing apical media once confluent (approximately 3-4 days). Cells were maintained with basolateral media only, changing daily, for 4-5 weeks (depending on schedule) until testing.

Ion Transport Analysis. Once mature, select inserts were pretreated with VX809 (3 M), VX661 (3 M), and/or VX445 (3 M) for 48 h prior to study. Inserts were removed from media and rinsed of any pre-treatment drugs, then mounted in Ussing chambers and studied as previously described (Brewington J J, Filbrandt E T, LaRosa F J, 3rd, Ostmann A J, Strecker L M, Szczesniak R D, Clancy J P. Detection of CFTR function and modulation in primary human nasal cell spheroids. Journal of cystic fibrosis: official journal of the European Cystic Fibrosis Society 2017; 17: 26-33). All studies were performed in an asymmetric chloride ringer buffer, producing a basolateral-to-apical Cl-secretory gradient. Under voltage-clamp conditions, cells were treated apically with 100 M Amiloride to block ENaC and sodium transport. Forskolin (10 μM) and IBMX (100 M) were added in both compartments to increase cAMP and stimulate CFTR. VX770 (1 μM) was added apically to potentiate CFTR. Finally, CFTR Inhibitor-172 (10 μM) was added to the apical compartment to block CFTR currents. Short-circuit current (Isc) and resistance were measured using Acquire and Analyze 2.3 software.

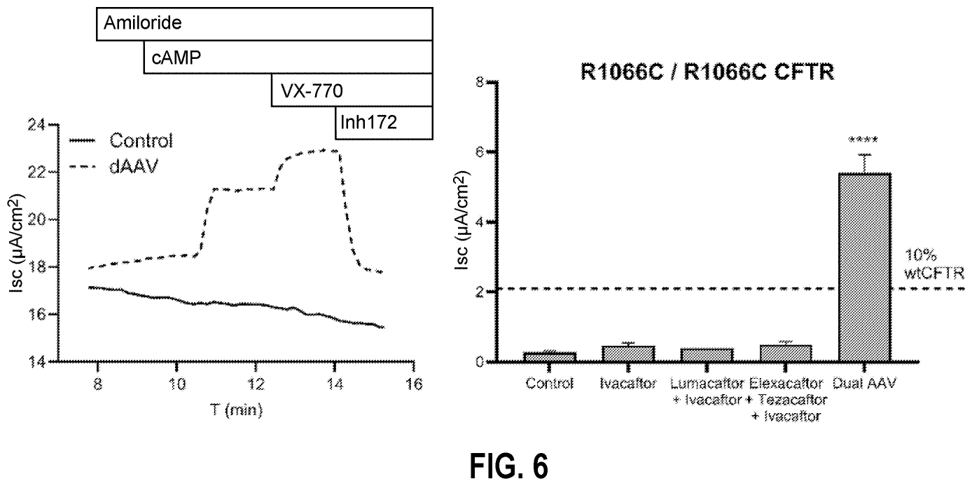

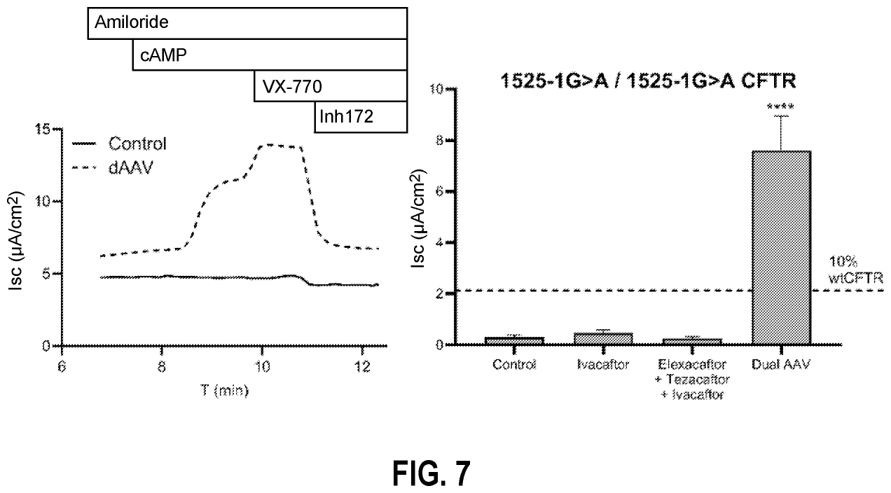

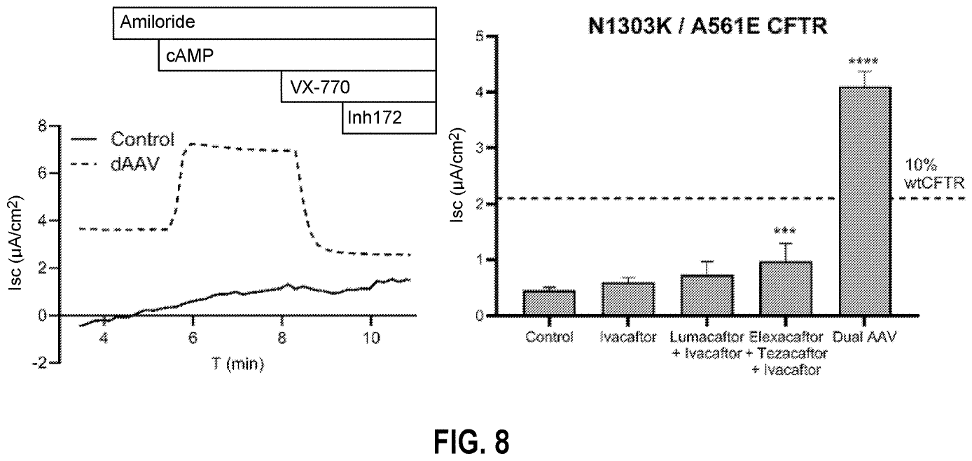

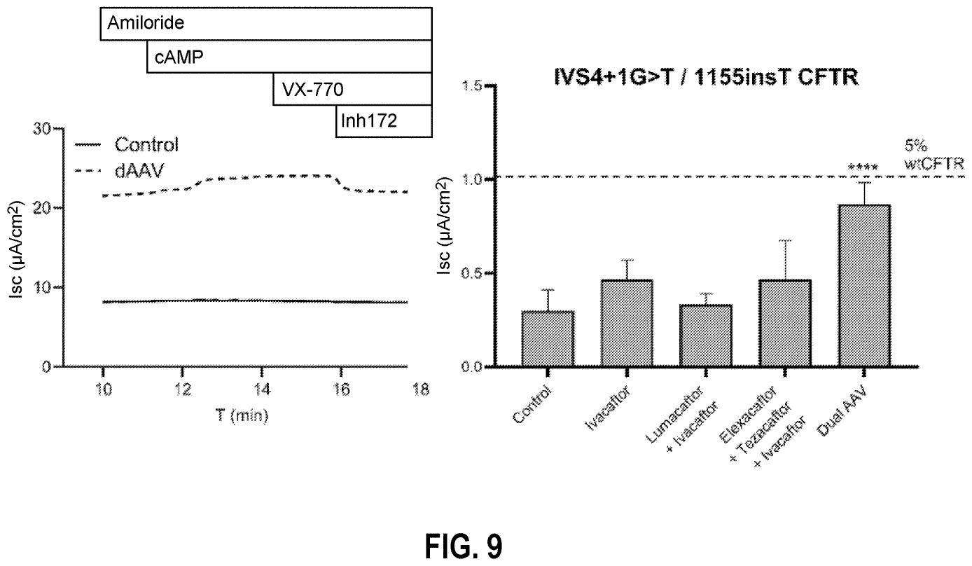

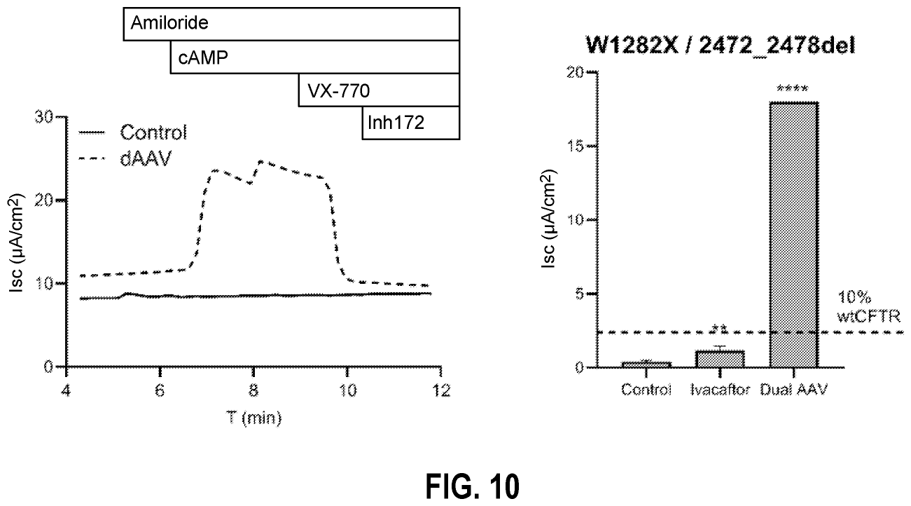

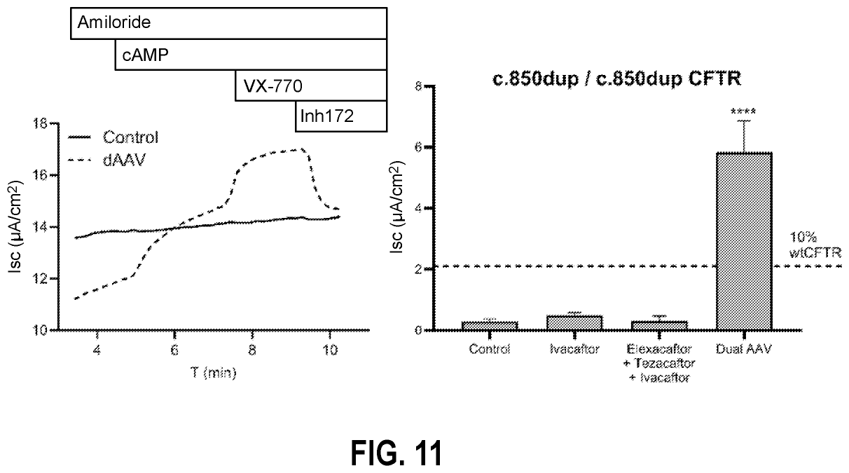

FIGS. 6-11 show cellular response data in human nasal epithelial (HNE) cultures from six individuals with cystic fibrosis (CF) without CFTR modulator access. CFTR function >10% of wild-type is easily achieved in five of six subjects, with a statistically significant response to dAAV still present in the sixth subject. For all aggregate data, n=4 inserts. **p<0.01; ***p<0.001; ****p<0.0001 by one-way ANOVA with Dunnet's multiple comparisons test against control samples.

### REFERENCES

1. Rang C, Keating D, Wilson J, Kotsimbos T. Re-imagining cystic fibrosis care: next generation thinking. Eur Respir J. 2020 May 27; 55(5):1902443. doi: 10.1183/13993003.02443-2019. PMID: 32139465.
2. Rosenfeld M A, Yoshimura K, Trapnell B C, Yoneyama K, Rosenthal E R, Dalemans W, Fukayama M, Bargon J, Stier L E, Stratford-Perricaudet L, et al. In vivo transfer of the human cystic fibrosis transmembrane conductance regulator gene to the airway epithelium. Cell. 1992 Jan. 10; 68(1):143-55. doi: 10.1016/0092-8674(92)90213-v. PMID: 1370653.
3. Marshall E. Gene therapy death prompts review of adenovirus vector. Science. 1999 Dec. 17; 286(5448):2244-5. doi: 10.1126/science.286.5448.2244. PMID: 10636774.
4. Amit C Nathwani, Ulrike Reiss, Edward Tuddenham, Pratima Chowdary, Jenny McIntosh, Anne Riddell, Jun Pie, Johnny N Mahlangu, Michael Recht, Yu-Min Shen, Kathleen G. Halka, Michael M Meagher, Arthur W. Nienhuis, Andrew M. Davidoff, Sarah Mangles, Chris L Morton, Zhou Junfang, Vlad Calin Radulescu; Adeno-Associated Mediated Gene Transfer for Hemophilia B:8 Year Follow up and Impact of Removing “Empty Viral Particles” on Safety and Efficacy of Gene Transfer. Blood 2018; 132 (Supplement 1): 491. doi: https://doi.org/10.1182/blood-2018-99-118334
5. George L A, Monahan P E, Eyster M E, Sullivan S K, Ragni M V, Croteau S E, Rasko J E J, Recht M, Samelson-Jones B J, MacDougall A, Jaworski K, Noble R, Curran M, Kuranda K, Mingozzi F, Chang T, Reape K Z, Anguela X M, High K A. Multiyear Factor VIII Expression after AAV Gene Transfer for Hemophilia A. N Engl J Med. 2021 Nov. 18; 385(21):1961-1973. doi: 10.1056/NEJMoa2104205. PMID: 34788507; PMCID: PMC8672712.
6. Flotte T R, Afione S A, Solow R, Drumm M L, Markakis D, Guggino W B, Zeitlin P L, Carter B J. Expression of the cystic fibrosis transmembrane conductance regulator from a novel adeno-associated virus promoter. J Biol Chem. 1993 Feb. 15; 268(5):3781-90. PMID: 7679117.
7. Flotte T R, Afione S A, Conrad C, McGrath S A, Solow R, Oka H, Zeitlin P L, Guggino W B, Carter B J. Stable in vivo expression of the cystic fibrosis transmembrane conductance regulator with an adeno-associated virus vector. Proc Natl Acad Sci USA. 1993 Nov. 15; 90(22):10613-7. doi: 10.1073/pnas.90.22.10613. PMID: 7504271; PMCID: PMC47827.
8. Zhang L, Wang D, Fischer H, Fan P D, Widdicombe J H, Kan Y W, Dong J Y. Efficient expression of CFTR function with adeno-associated virus vectors that carry shortened CFTR genes. Proc Natl Acad Sci USA. 1998 Aug. 18; 95(17):10158-63. doi: 10.1073/pnas.95.17.10158. PMID: 9707617; PMCID: PMC21478.
9. Ostedgaard L S, Rokhlina T, Karp P H, Lashmit P, Afione S, Schmidt M, Zabner J, Stinski M F, Chiorini J A, Welsh M J. A shortened adeno-associated virus expression cassette for CFTR gene transfer to cystic fibrosis airway epithelia. Proc Natl Acad Sci USA. 2005 Feb. 22; 102(8):2952-7. doi: 10.1073/pnas.0409845102. Epub 2005 Feb. 9. PMID: 15703296; PMCID: PMC549485.
10. Steines B, Dickey D D, Bergen J, Excoffon K J, Weinstein J R, Li X, Yan Z, Abou Alaiwa M H, Shah V S, Bouzek D C, Powers L S, Gansemer N D, Ostedgaard L S, Engelhardt J F, Stoltz D A, Welsh M J, Sinn P L, Schaffer D V, Zabner J. CFTR gene transfer with AAV improves early cystic fibrosis pig phenotypes. JCI Insight. 2016 Sep. 8; 1(14):e88728. doi: 10.1172/jci.insight.88728. PMID: 27699238; PMCID: PMC5033908.
11. Wagner J A, Nepomuceno I B, Messner A H, Moran M L, Batson E P, Dimiceli S, Brown B W, Desch J K, Norbash A M, Conrad C K, Guggino W B, Flotte T R, Wine J J, Carter B J, Reynolds T C, Moss R B, Gardner P. A phase II, double-blind, randomized, placebo-controlled clinical trial of tgAAVCF using maxillary sinus delivery in patients with cystic fibrosis with antrostomies. Hum Gene Ther. 2002 Jul. 20; 13(11):1349-59. doi: 10.1089/104303402760128577. PMID: 12162817.
12. The Lancet Respiratory Medicine. Cystic fibrosis treatment: hope for all?Lancet Respir Med. 2022 December; 10(12):1099. doi: 10.1016/S2213-2600(22)00447-7. Epub 2022 November 16. PMID: 36402149.
13. Liu X, Luo M, Zhang L N, Yan Z, Zak R, Ding W, Mansfield S G, Mitchell L G, Engelhardt J F. Spliceosome-mediated RNA trans-splicing with recombinant adeno-associated virus partially restores cystic fibrosis transmembrane conductance regulator function to polarized human cystic fibrosis airway epithelial cells. Hum Gene Ther. 2005 September; 16(9):1116-23. doi: 10.1089/hum.2005.16.1116. PMID: 16149910.
14. Song Y, Lou H H, Boyer J L, Limberis M P, Vandenberghe L H, Hackett N R, Leopold P L, Wilson J M, Crystal R G. Functional cystic fibrosis transmembrane conductance regulator expression in cystic fibrosis airway epithelial cells by AAV6.2-mediated segmental trans-splicing. Hum Gene Ther. 2009 March; 20(3):267-81. doi: 10.1089/hum.2008.173. PMID: 19257851; PMCID: PMC2855253.
15. Chew W L, Tabebordbar M, Cheng J K, Mali P, Wu E Y, Ng A H, Zhu K, Wagers A J, Church G M. A multifunctional AAV-CRISPR-Cas9 and its host response. Nat Methods. 2016 October; 13(10):868-74. doi: 10.1038/nmeth.3993. Epub 2016 Sep. 5. PMID: 27595405; PMCID: PMC5374744.
16. Chen, Y. X., Zhi, S. Y., Liu, W. L., Wen, J. K., Hu, S. H., Cao, T. Q., Sun, H. W., Li, Y., Huang, L., Liu, Y. Z., Liang, P. P., Huang, J. J., Development of Highly Efficient Dual-AAV Split Adenosine Base Editor for In Vivo Gene Therapy. Small Methods 2020, 4, 2000309. https://doi.org/10.1002/smtd.202000309
17. Duan D, Sharma P, Yang J, Yue Y, Dudus L, Zhang Y, Fisher K J, Engelhardt J F. Circular intermediates of recombinant adeno-associated virus have defined structural characteristics responsible for long-term episomal persistence in muscle tissue. J Virol. 1998 November; 72(11):8568-77. doi: 10.1128/JVI.72.11.8568-8577.1998. Erratum in: J Virol 1999 January; 73(1):861. PMID: 9765395; PMCID: PMC110267.
18. Zhu F X, Liu Z L, Qu H G, Chi X Y. [Post-translational ligation and function of dual-vector transferred split CFTR gene]. Yao Xue Xue Bao. 2010 January; 45(1):60-5. Chinese. PMID: 21351451.
19. Zhu F, Gong X, Liu Z, Yang S, Qu H, Chi X. [Post-translational ligation of split CFTR severed before TMD2 and its chloride channel function]. Sheng Wu Gong Cheng Xue Bao. 2010 December; 26(12):1710-6. Chinese. PMID: 21387835.
20. Lv J, Wang H, Cheng X, Chen Y, Wang D, Zhang L, Cao Q, Tang H, Hu S, Gao K, Xun M, Wang J, Wang Z, Zhu B, Cui C, Gao Z, Guo L, Yu S, Jiang L, Yin Y, Zhang J, Chen B, Wang W, Chai R, Chen Z Y, Li H, Shu Y. AAV1-hOTOF gene therapy for autosomal recessive deafness 9: a single-arm trial. Lancet. 2024 May 25; 403(10441):2317-2325. doi: 10.1016/S0140-6736(23)02874-X. Epub 2024 Jan. 24. Erratum in: Lancet. 2024 May 25; 403(10441):2292. PMID: 38280389
21. Tang H, Wang H, Wang S, Hu S W, Lv J, Xun M, Gao K, Wang F, Chen Y, Wang D, Wang W, Li H, Shu Y. Hearing of Otof-deficient mice restored by trans-splicing of N- and C-terminal otoferlin. Hum Genet. 2023 February; 142(2):289-304. doi: 10.1007/s00439-022-02504-2. Epub 2022 Nov. 16. PMID: 36383253.
22. Tornabene P, Trapani I, Minopoli R, Centrulo M, Lupo M, de Simone S, Tiberi P, Dell'Aquila F, Marrocco E, Iodice C, Iuliano A, Gesualdo C, Rossi S, Giaquinto L, Albert S, Hoyng C B, Polishchuk E, Cremers F P M, Surace E M, Simonelli F, De Matteis M A, Polishchuk R, Auricchio A. Intein-mediated protein trans-splicing expands adeno-associated virus transfer capacity in the retina. Sci Transl Med. 2019 May 15; 11(492):eaav4523. doi: 10.1126/scitranslmed.aav4523. PMID: 31092694; PMCID: PMC6863751.
23. Esposito F, Lyubenova H, Tornabene P, Auricchio S, Iuliano A, Nusco E, Merlin S, Olgasi C, Manni G, Gargaro M, Fallarino F, Follenzi A, Auricchio A. Liver gene therapy with intein-mediated F8 trans-splicing corrects mouse haemophilia A. EMBO Mol Med. 2022 Jun. 8; 14(6):e15199. doi: 10.15252/emmm.202115199. Epub 2022 May 2. PMID: 35491676; PMCID: PMC9174883.
24. Koblan L W, Erdos M R, Wilson C, Cabral W A, Levy J M, Xiong Z M, Tavarez U L, Davison L M, Gete Y G, Mao X, Newby G A, Doherty S P, Narisu N, Sheng Q, Krilow C, Lin C Y, Gordon L B, Cao K, Collins F S, Brown J D, Liu D R. In vivo base editing rescues Hutchinson-Gilford progeria syndrome in mice. Nature. 2021 January; 589(7843):608-614. doi: 10.1038/s41586-020-03086-7. Epub 2021 Jan. 6. PMID: 33408413; PMCID: PMC7872200.
25. Zabner J, Seiler M, Walters R, Kotin R M, Fulgeras W, Davidson B L, Chiorini J A. Adeno-associated virus type 5 (AAV5) but not AAV2 binds to the apical surfaces of airway epithelia and facilitates gene transfer. J Virol. 2000 April; 74(8):3852-8. doi: 10.1128/jvi.74.8.3852-3858.2000. PMID: 10729159; PMCID: PMC111893.
26. Limberis M P, Vandenberghe L H, Zhang L, Pickles R J, Wilson J M. Transduction efficiencies of novel AAV vectors in mouse airway epithelium in vivo and human ciliated airway epithelium in vitro. Mol Ther. 2009 February; 17(2):294-301. doi: 10.1038/mt.2008.261. Epub 2008 Dec. 9. PMID: 19066597; PMCID: PMC2835069.
27. Walters R W, Yi S M, Keshavjee S, Brown K E, Welsh M J, Chiorini J A, Zabner J. Binding of adeno-associated virus type 5 to 2,3-linked sialic acid is required for gene transfer. J Biol Chem. 2001 Jun. 8; 276(23):20610-6. doi: 10.1074/jbc.M101559200. Epub 2001 Mar. 21. PMID: 11262413.
28. Duncan G A, Kim N, Colon-Cortes Y, Rodriguez J, Mazur M, Birket S E, Rowe S M, West N E, Livraghi-Butrico A, Boucher R C, Hanes J, Aslanidi G, Suk J S. An Adeno-Associated Viral Vector Capable of Penetrating the Mucus Barrier to Inhaled Gene Therapy. Mol Ther Methods Clin Dev. 2018 Mar. 22; 9:296-304. doi: 10.1016/j.omtm.2018.03.006. PMID: 30038933; PMCID: PMC6054694.
29. Guo M, Morley M P, Jiang C, Wu Y, Li G, Du Y, Zhao S, Wagner A, Cakar A C, Kouril M, Jin K, Gaddis N, Kitzmiller J A, Stewart K, Basil M C, Lin S M, Ying Y, Babu A, Wikenheiser-Brokamp K A, Mun K S, Naren A P, Clair G, Adkins J N, Pryhuber G S, Misra R S, Aronow B J, Tickle T L, Salomonis N, Sun X, Morrisey E E, Whitsett J A; NHLBI LungMAP Consortium; Xu Y. Guided construction of single cell reference for human and mouse lung. Nat Commun. 2023 Jul. 29; 14(1):4566. doi: 10.1038/s41467-023-40173-5. PMID: 37516747; PMCID: PMC10387117.
30. Plasschaert L W, Zilionis R, Choo-Wing R, Savova V, Knehr J, Roma G, Klein A M, Jaffe A B. A single-cell atlas of the airway epithelium reveals the CFTR-rich pulmonary ionocyte. Nature. 2018 August; 560(7718):377-381. doi: 10.1038/s41586-018-0394-6. Epub 2018 Aug. 1. PMID: 30069046; PMCID: PMC6108322.
31. Montoro D T, Haber A L, Biton M, Vinarsky V, Lin B, Birket S E, Yuan F, Chen S, Leung H M, Villoria J, Rogel N, Burgin G, Tsankov A M, Waghray A, Slyper M, Waldman J, Nguyen L, Dionne D, Rozenblatt-Rosen O, Tata P R, Mou H, Shivaraju M, Bihler H, Mense M, Tearney G J, Rowe S M, Engelhardt J F, Regev A, Rajagopal J. A revised airway epithelial hierarchy includes CFTR-expressing ionocytes. Nature. 2018 August; 560(7718):319-324. doi: 10.1038/s41586-018-0393-7. Epub 2018 Aug. 1. PMID: 30069044; PMCID: PMC6295155.
32. van Lieshout L P, Domm J M, Rindler T N, Frost K L, Sorensen D L, Medina S J, Booth S A, Bridges J P, Wootton S K. A Novel Triple-Mutant AAV6 Capsid Induces Rapid and Potent Transgene Expression in the Muscle and Respiratory Tract of Mice. Mol Ther Methods Clin Dev. 2018 Apr. 14; 9:323-329. doi: 10.1016/j.omtm.2018.04.005. PMID: 30038936; PMCID: PMC6054702.
33. Brewington J J, Filbrandt E T, LaRosa F J 3rd, Moncivaiz J D, Ostmann A J, Strecker L M, Clancy J P. Brushed nasal epithelial cells are a surrogate for bronchial epithelial CFTR studies. JCI Insight. 2018 Jul. 12; 3(13):e99385. doi: 10.1172/jci.insight.99385. PMID: 29997283; PMCID: PMC6124537.
34. Marquez Loza L I, Cooney A L, Dong Q, Randak C O, Rivella S, Sinn P L, McCray P B Jr. Increased CFTR expression and function from an optimized lentiviral vector for cystic fibrosis gene therapy. Mol Ther Methods Clin Dev. 2021 Feb. 27; 21:94-106. doi: 10.1016/j.omtm.2021.02.020. PMID: 33768133; PMCID: PMC7973238.
35. Sun X, Sui H, Fisher J T, Yan Z, Liu X, Cho H J, Joo N S, Zhang Y, Zhou W, Yi Y, Kinyon J M, Lei-Butters D C, Griffin M A, Naumann P, Luo M, Ascher J, Wang K, Frana T, Wine J J, Meyerholz D K, Engelhardt J F. Disease phenotype of a ferret CFTR-knockout model of cystic fibrosis. J Clin Invest. 2010 September; 120(9):3149-60. doi: 10.1172/JCI43052. Epub 2010 August 25. PMID: 20739752; PMCID: PMC2929732.
36. Rogers C S, Stoltz D A, Meyerholz D K, Ostedgaard L S, Rokhlina T, Taft P J, Rogan M P, Pezzulo A A, Karp P H, Itani O A, Kabel A C, Wohlford-Lenane C L, Davis G J, Hanfland R A, Smith T L, Samuel M, Wax D, Murphy C N, Rieke A, Whitworth K, Uc A, Starner T D, Brogden K A, Shilyansky J, McCray P B Jr, Zabner J, Prather R S, Welsh M J. Disruption of the CFTR gene produces a model of cystic fibrosis in newborn pigs. Science. 2008 Sep. 26; 321(5897):1837-41. doi: 10.1126/science.1163600. PMID: 18818360; PMCID: PMC2570747.
37. Mall M, Grubb B R, Harkema J R, O'Neal W K, Boucher R C. Increased airway epithelial Na+ absorption produces cystic fibrosis-like lung disease in mice. Nat Med. 2004 May; 10(5):487-93. Epub 2004 Apr. 11. PubMed PMID: 15077107.
38. Tornabene P, Trapani I, Centrulo M, Marrocco E, Minopoli R, Lupo M, Iodice C, Gesualdo C, Simonelli F, Surace E M, Auricchio A. Inclusion of a degron reduces levels of undesired inteins after AAV-mediated protein trans-splicing in the retina. Mol Ther Methods Clin Dev. 2021 Oct. 19; 23:448-459. doi: 10.1016/j.omtm.2021.10.004. PMID: 34786437; PMCID: PMC8571531
39. Charan J, Kantharia N D. How to calculate sample size in animal studies?J Pharmacol Pharmacother. 2013 October; 4(4):303-6. doi: 10.4103/0976-500X.119726. PMID: 24250214; PMCID: PMC3826013.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

1. A paired nucleic acid construct system for delivery of a human cystic fibrosis transmembrane conductance regulator (CFTR) protein to an individual in need thereof, the system comprising:
a. a first construct comprising a first portion (N-terminus) of a human CFTR gene fused to Rhodothermus marinus intein-N cDNA (CFTR-N-inteinN); and
b. a second construct comprising a second portion (C-terminus) of the human CFTR gene fused to Rhodothermus marinus intein-C cDNA (inteinC-CFTR-C);
wherein the first portion of the human CFTR gene comprises the N terminal portion of the human CFTR gene and the second portion of the human CFTR gene comprises the carboxy terminal portion of the human CFTR gene.

2. The paired nucleic acid construct system of claim 1, wherein the first portion of the human CFTR gene encodes for a protein having at least 90%, or at least 95% sequence identity to SEQ ID NO: 6.

3. The paired nucleic acid construct system of claim, wherein the second portion of the human CFTR gene encodes for a protein having at least 90%, or at least 95% sequence identity to SEQ ID NO: 8.

4. The paired nucleic acid construct system of claim 2, wherein the first portion and the second portion of the human CFTR gene total a complete human CFTR gene.

5. The paired nucleic acid construct system of claim 1, wherein the first portion of the human CFTR gene and the second portion of the human CFTR gene are capable of being fused and expressing a functional CFTR protein.

6. The paired nucleic acid construct system of claim 5, wherein the functional CFTR protein comprises a CFTR R domain.

7. The paired nucleic acid construct system of claim 1, further comprising
a. a first AAV vector operatively linked to the first construct; and
b. a second AAV vector operatively linked to the second construct.

8. The paired nucleic acid construct system of claim 7, wherein the first AAV vector and/or the second AAV vector is an AAV serotype 6 (AAV6).

9. A method of treating an individual having Cystic Fibrosis (CF) comprising administering the paired nucleic acid construct system of claim 1 to the individual.

10. The method of claim 9, wherein the paired nucleic acid construct system is contacted with airway epithelial cells of the individual.

11. The method of claim 9, wherein the administering restores CFTR activity in airway cells of the individual.

12. The method of claim 9, wherein the individual has a class 1 mutation.

13. The method of claim 12, wherein the class I mutation is G542X, R553X, and/or W1282X.

14. The method of claim 9, wherein the individual has one or both of a F5O8 deletion and a 2184delA mutation.

15. The method of claim 9, wherein the individual does not respond to a CFTR modulator drug.

16. The method of claim 15, wherein the CFTR modulator drug is TRIKAFTA (elexacaftor/tezacaftor/ivacaftor).

17. The method of claim 9, wherein the paired nucleic acids of the system are administered simultaneously.

18. The method of claim 9, wherein the paired nucleic acids of the system are administered sequentially.

19. (canceled)

20. (canceled)

21. (canceled)

22. A cell, or plurality of cells that express the paired nucleic acid construct system according to claim 1.

23. A pharmaceutical composition comprising the paired nucleic acid construct system of claim 1 and a pharmaceutically acceptable vehicle.


SEQUENCE LISTING (28 sequences)

SEQ ID NO: 1 - DNA, 309 bp, Rhodothermus marinus
  source 1..309: mol_type = genomic DNA
tgtctggctggcgatactctcattaccctggccgatggacgacgagtgcctattagagaactggtgtcacagcagaatttttccgtgtgggctctgaatcctcagacttaccgcctggagagggctagagtgagtagagctttctgtaccggcatcaaacctgtgtaccgcctcaccactagactggggagatccattagggccactgccaaccaccgatttctcacacctcagggctggaaacgagtcgatgaactccagcctggagattacctggctctgcctaggagaatccctactgcctcctga

SEQ ID NO: 2 - protein, 102 aa, Rhodothermus marinus
CLAGDTLITLADGRRVPIRELVSQQNFSVWALNPQTYRLERARVSRAFCTGIKPVYRLTTRLGRSIRATANHRFLTPQGWKRVDELQPGDYLALPRRIPTAS

SEQ ID NO: 3 - DNA, 159 bp, Rhodothermus marinus
  source 1..159: mol_type = genomic DNA
atggcggcggcgtgcccggaactgcgtcagctggcgcagagcgatgtgtattgggatccgattgtgagcattgaaccggatggcgtggaagaagtgtttgatctgaccgtgccgggcccgcataactttgtggcgaacgatattattgcgcataactct

SEQ ID NO: 4 - protein, 53 aa, Rhodothermus marinus
MAAACPELRQLAQSDVYWDPIVSIEPDGVEEVFDLTVPGPHNFVANDIIAHNS

SEQ ID NO: 5 - DNA, 2514 bp, Homo sapiens
  source 1..2514: mol_type = genomic DNA
atgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatggtgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcttttttgatgatatggag

SEQ ID NO: 6 - protein, 838 aa, Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDME

SEQ ID NO: 7 - DNA, 1926 bp, Homo sapiens
  source 1..1926: mol_type = genomic DNA
agcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctt

SEQ ID NO: 8 - protein, 642 aa, Homo sapiens
SIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 9 - DNA, 808 bp, synthetic construct
  source 1..808: mol_type = other DNA
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacag

SEQ ID NO: 10 - DNA, 72 bp, synthetic construct
  source 1..72: mol_type = other DNA
atggactacaaagaccatgacggtgattataaagatcatgacatcgattacaaggatgacgatgacaagctt

SEQ ID NO: 11 - protein, 24 aa, synthetic construct
MDYKDHDGDYKDHDIDYKDDDDKL

SEQ ID NO: 12 - DNA, 2895 bp, synthetic construct
  source 1..2895: mol_type = other DNA
atggactacaaagaccatgacggtgattataaagatcatgacatcgattacaaggatgacgatgacaagcttatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatggtgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcttttttgatgatatggagtgtctggctggcgatactctcattaccctggccgatggacgacgagtgcctattagagaactggtgtcacagcagaatttttccgtgtgggctctgaatcctcagacttaccgcctggagagggctagagtgagtagagctttctgtaccggcatcaaacctgtgtaccgcctcaccactagactggggagatccattagggccactgccaaccaccgatttctcacacctcagggctggaaacgagtcgatgaactccagcctggagattacctggctctgcctaggagaatccctactgcctcctga

SEQ ID NO: 13 - protein, 964 aa, synthetic construct
MDYKDHDGDYKDHDIDYKDDDDKLMQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMECLAGDTLITLADGRRVPIRELVSQQNFSVWALNPQTYRLERARVSRAFCTGIKPVYRLTTRLGRSIRATANHRFLTPQGWKRVDELQPGDYLALPRRIPTAS

SEQ ID NO: 14 - DNA, 182 bp, synthetic construct
  source 1..182: mol_type = other DNA
acgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaa

SEQ ID NO: 15 - DNA, 429 bp, synthetic construct
  source 1..429: mol_type = other DNA
aaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 16 - DNA, 130 bp, Adeno-associated virus serotype 2
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 17 - DNA, 297 bp, synthetic construct
  source 1..297: mol_type = other DNA
tgtagttaatgattaacccgccatgctacttatctacctgcggccgcggaatgtgtgtcagttagggtgtggaaagtccccaggctccccagcaggcagaagtatgcaaagcatgcatctcaattagtcagcaaccagtcccggtctcctcccatgcatgtcaatattggccattagccatattattcattggttatatagcataaatcaatattggctattggccattgcatacgttgtatctatatcataatatgtacatttatattggctcatgtccaatatgaccgccatgtt

SEQ ID NO: 18 - DNA, 28 bp, synthetic construct
  source 1..28: mol_type = other DNA
gggtaccgaagccgctagcgctaccggt

SEQ ID NO: 19 - DNA, 10 bp, synthetic construct
  source 1..10: mol_type = other DNA
agaattaacc

SEQ ID NO: 20 - DNA, 38 bp, synthetic construct
  source 1..38: mol_type = other DNA
ctcgagctcgatgagtttggacaaaccacaactagaat

SEQ ID NO: 21 - DNA, 82 bp, Simian virus 40
  source 1..82: mol_type = genomic DNA
gcagtgaaaaaaatgctttatttgtgaaatttgtgatgctattgctttatttgtaaccattataagctgcaataaacaagtt

SEQ ID NO: 22 - DNA, 182 bp, synthetic construct
  source 1..182: mol_type = other DNA
aacaacaacaattgcattcattttatgtttcaggttcagggggaggtgtgggaggttttttaaagcaagtaaaacctctacaaatgtggtagcggccgcggaatgtgtgtcagtaaaacctctacaaatgtggtagcggccgcaggtagataagtagcatggcgggttaatcattaactaca

SEQ ID NO: 23 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 24 - DNA, 2200 bp, synthetic construct
  source 1..2200: mol_type = other DNA
ccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgcc

SEQ ID NO: 25 - DNA, 30 bp, Influenza virus A
  source 1..30: mol_type = genomic DNA
tacccatacgatgttccagattacgcttag

SEQ ID NO: 26 - protein, 9 aa, Influenza virus A
YPYDVPDYA

SEQ ID NO: 27 - DNA, 2115 bp, synthetic construct
  source 1..2115: mol_type = other DNA
atggcggcggcgtgcccggaactgcgtcagctggcgcagagcgatgtgtattgggatccgattgtgagcattgaaccggatggcgtggaagaagtgtttgatctgaccgtgccgggcccgcataactttgtggcgaacgatattattgcgcataactctagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttacccatacgatgttccagattacgcttag

SEQ ID NO: 28 - protein, 704 aa, synthetic construct
MAAACPELRQLAQSDVYWDPIVSIEPDGVEEVFDLTVPGPHNFVANDIIAHNSSIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRLYPYDVPDYA